(19) United States
(12) Patent Application Publication
(10) Pub. No.: US 2025/0313965 A1
(43) Pub. Date: Oct. 9, 2025

(54) ANAEROBIC DIGESTION SYSTEM AND PROCESS FOR POULTRY LITTER TREATMENT WITH WATER RECIRCULATION

(71) Applicants: University of Idaho, Moscow, ID (US); BOARD OF TRUSTEES OF THE UNIVERSITY OF ARKANSAS, Little Rock, AR (US)

(72) Inventors: Jun Zhu, Fayetteville, AR (US); Sarah Wu, Moscow, ID (US)

(73) Assignees: University of Idaho, Moscow, ID (US); BOARD OF TRUSTEES OF THE UNIVERSITY OF ARKANSAS, Little Rock, AR (US)

(21) Appl. No.: 19/170,930

(22) Filed: Apr. 4, 2025

Related U.S. Application Data
(60) Provisional application No. 63/575,818, filed on Apr. 7, 2024.

(51) Int. Cl. C25B 1/18; C02F 1/44; C02F 11/04; C02F 103/20; C12M 1/00; C12M 1/107; C12P 5/02; C25B 9/00; C25B 11/042

(52) U.S. Cl. CPC C25B 1/18; C02F 1/445; C02F 11/04; C12M 21/04; C12M 43/00; C12P 5/023; C25B 9/00; C25B 11/042; C02F 2103/20

(57) ABSTRACT

The invention relates to a system and process for digesting poultry litter. In general, in a first aspect, a poultry litter digester includes an anaerobic digestion reactor configured to digest poultry litter to produce an effluent and a biogas, and an electrolytic reactor having at least one magnesium anode, where the electrolytic reactor is configured to precipitate struvite from the effluent.

### CROSS-REFERENCE TO RELATED APPLICATIONS

This application claims the benefit of U.S. Provisional Patent Application No. 63/575,818 filed Apr. 7, 2024, and incorporates said provisional application by reference in its entirety into this document as if fully set out at this point.

### GOVERNMENT LICENSE RIGHTS

This invention was made with government support under Grant No. 2019-67021-29945 awarded by the United States Department of Agriculture, National Institute of Food and Agriculture (USDA/NIFA). The government has certain rights in the invention.

### BACKGROUND OF THE INVENTION

### 1. Field of the Invention

This invention generally relates to poultry production and, more particularly, to the treatment of poultry litter.

### 2. Description of the Related Art

Increasing consumer demand for poultry products has led to intensive poultry farming operations globally. Fast growth of the poultry industry has resulted in a significant increase in waste production and resulting disposal issues, particularly for the large amount of chicken manure produced in the chicken houses on farms. In addition to creating nuisance odors, the accumulation of poultry litter poses serious environmental pollution threats to air, soil, and water resources (e.g., through excess nutrient runoff in watersheds) in the absence of adequate treatment or disposal measures. Poultry producers are ill-equipped to perform on-site treatment of poultry litter, and poultry production costs are increased when poultry litter must be transported to an offsite location for waste processing or disposal.

Several techniques have been proposed to address the issue of poultry waste, including composting, direct combustion, pelletization, and anaerobic digestion. Of the existing technologies, anaerobic digestion provides the best recycling. Anaerobic digestion is a natural process that uses microorganisms to break down biodegradable material, such as livestock manure, municipal wastewater solids and food waste, in the absence of oxygen. One downside of anaerobic digestion is that it requires liquids to work properly (roughly 90% to 95% moisture content), and chicken litter has a low moisture content. Poultry producers also wish to avoid large quantities of litter, which anaerobic digestion generates. Further, ammonia toxicity is a common inhibition problem during anaerobic digestion of poultry litter.

In terms of economics, current poultry litter processing is expensive and recovers little to no value from the waste. Notably, the over 598 million metric tons (MMT) of waste products generated by large-scale farming operations contains about 20.5 MMT of recoverable phosphorus (P₂O₅). This amount of recoverable phosphorous from poultry litter alone could reduce depletion of natural phosphate reserves in the United States by substituting up to 82% of the country's total P₂O₅ consumption, which is about 25 MMT. It is therefore desirable to recover the high concentrations of phosphate (PO₄³⁻) and ammonium nitrogen (NH₃—N) which are typically present in poultry manure effluents. Transforming poultry manure into a value-added product like struvite (MgNH₄PO₄·6H₂O) could provide a resource for crop production without significant environmental harm. Struvite is a white crystalline substance that is spontaneously formed under alkaline conditions when magnesium (Mg²⁺) is added to an aqueous solution containing NH₄⁺ and PO₄³⁻ ions. It can be applied directly to the soil and is generally regarded as a premium-grade, slow-release fertilizer that is less prone to leaching since it is only marginally soluble in water and soil solution. Struvite can be used to grow crops such as corn, soybeans, and rice. In this respect, struvite performs just as well—and in some cases better than—mined phosphate which is a finite resource prone to price fluctuations. The two major processes for NH₃—N and PO₄³⁻ recovery through struvite precipitation are chemical struvite precipitation and electrochemical struvite precipitation.

Chemical struvite precipitation processes have been applied to various waste streams (industrial wastewater, municipal wastewater, aerobic digestion calf/swine manure) and have been successfully implemented at different pilot- and full-scale units. However, this struvite precipitation technology is not without limitations. First, for struvite precipitation to occur, the molar ratios of Mg²⁺, NH₄⁺, and PO₄³⁻ should be at least 1:1:1 according to the stoichiometric composition of struvite, and the solution should be in the alkaline condition. Since animal manure normally contains much higher nitrogen than Mg²⁺, adding magnesium salt to the solution is always required. In chemical struvite precipitation, magnesium salts such as Mg(OH)₂, MgO, MgCl₂, MgCO₃, and MgSO₄ are added to a wastewater solution containing NH⁴⁺ and PO₄³⁻ ions. This practice unfortunately leads to high levels of Cl⁻ and SO₄²⁻ in the treated liquid, which become an environmental concern when the liquid is discharged. The alkaline condition necessary to precipitate struvite is also normally established by mixing NaOH into the liquid to raise pH, and this pH adjustment both increases treatment cost and adds complexity to the operation of the treatment process.

In contrast, electrochemical struvite precipitation involves the release of Mg²⁺ ions into solution by applying a small amount of electric current to a Mg sacrificial electrode. Electrochemical struvite precipitation using a Mg sacrificial anode has several advantages over chemical struvite precipitation. For instance, the process can be easily automated, does not require the addition of chemicals for pH adjustment, produces less sludge, can generate renewable fuel in the form of hydrogen gas, and reduces chemical oxygen demand (COD) levels in wastewater through oxidation and complexation.

Unfortunately, electrochemical struvite precipitation using a Mg sacrificial electrode is still at bench-scale. Several scale-up and design aspects still need to be addressed before full-scale applications can be developed for electrochemical struvite precipitation. Firstly, since one of the key goals of recovering PO₄³⁻ and NH₃—N from wastewater as struvite is to develop a commercially viable fertilizer product, the performance of a nutrient recovery process is measured by both the nutrient removal efficiency and the final product quality (crystal size, morphology, and purity). Bigger particles settle faster and bring fewer challenges during downstream and end-user applications such as handling, bulk density, filtration, granulation, tableting, drying times, blending, and storage, including product performance (dissolution and bioavailability). It is, therefore, necessary to tailor process design parameters and operating conditions to facilitate or promote crystal growth for enhanced product quality and recovery. In traditional reactor hardware for electrochemical struvite precipitation processes, magnetic stirrers and blade-type impellers promote contact between reactants, and such mixing devices may result in high levels of attrition and crystal breakage leading to excessive production of fines as strong mechanical interactions are naturally formed between the struvite crystals. Consequently, traditional designs are unsuitable for scale-up operations since large crystals with a narrow size distribution are required for efficient nutrient recovery and fertilizer application.

Secondly, majority of investigations for nutrient recovery via electrochemical struvite precipitation have been conducted using synthetic wastewater solutions. Process optimization data based on such studies are not suitable or credible for supporting process scale-up decisions since the chemical ion compositions of simulated solutions are vastly different from real high-strength agricultural wastewaters.

It is therefore desirable to replace existing poultry litter treatment techniques with systems and methods that can transform poultry litter to a valuable end product and support the sustainability of national agricultural and food systems.

Before proceeding to a detailed description of the invention, it should be noted and remembered that the description of the invention which follows, together with the accompanying drawings, should not be construed as limiting the invention to the examples (or embodiments) shown and described. Those skilled in the art to which the invention pertains will be able to devise other forms of this invention within the ambit of the appended claims.

### SUMMARY OF THE INVENTION

The invention relates to a system and process for digesting poultry litter. In general, in a first aspect, a poultry litter digester includes an anaerobic digestion reactor configured to digest poultry litter to produce an effluent and a biogas, and an electrolytic reactor having at least one magnesium anode, where the electrolytic reactor is configured to precipitate struvite from the effluent.

In an embodiment, the effluent includes Mg²⁺, NH₄⁺, and PO₄³⁻. In another embodiment, the biogas includes methane.

In an embodiment, the anaerobic digestion reactor is an anaerobic sequencing batch reactor, a plug flow reactor, an induced blanket reactor, an up flow anaerobic sludge blanket, a continuous stirred tank reactor, or an anaerobic filter and, more particularly, the anaerobic digestion reactor is the anaerobic sequencing batch reactor and, more particularly, a liquid anaerobic sequencing batch reactor that is configured to perform micro-aeration.

In an embodiment, the electrolytic reactor is a column air-lift electrolytic reactor or a dual-chamber reactor and, more particularly the electrolytic reactor is the column air-lift electrolytic reactor.

In an embodiment, the electrolytic reactor is further configured to produce a rejected water as the struvite is precipitated.

In an embodiment, the poultry litter digester also includes a water reclamation system configured to convert the rejected water from the electrolytic reactor into reclaimed water, and at least one pump configured to transfer the reclaimed water to the anaerobic digestion reactor.

In an embodiment, the water reclamation system is a forward osmosis reactor.

In another aspect, a method for digesting poultry litter includes the steps of preparing a substrate solution from poultry litter and a carbon-rich source, introducing the substrate solution into an anaerobic digestion reactor, performing anaerobic co-digestion on the substrate solution within the anaerobic digestion reactor to recover an effluent and a biogas, and routing the effluent to an electrolytic reactor, where the electrolytic reactor is configured to precipitate struvite from the effluent.

In an embodiment, the step of performing anaerobic co-digestion on the substrate solution involves introducing air or oxygen to the anaerobic digestion reactor.

In an embodiment, the air or oxygen is introduced to the anaerobic digestion reactor at an air supply rate of 25 mL per L of reactor working volume per day.

In an embodiment, the electrolytic reactor is further configured to produce a rejected water as the struvite is precipitated.

In an embodiment, the method further includes the steps of recycling the rejected water into a reclaimed water and pumping the reclaimed water to the anaerobic digestion reactor.

In an embodiment, the method further includes the step of drying the struvite.

In yet another aspect, a poultry litter digester includes an anaerobic digestion reactor configured to digest poultry litter to produce an effluent and a biogas, an electrolytic reactor having at least one magnesium anode, and a water reclamation system. The electrolytic reactor is configured to precipitate struvite from the effluent while producing a rejected water, and the water reclamation system is configured to convert the rejected water from the electrolytic reactor into reclaimed water.

In an embodiment, the anaerobic digestion reactor is configured to perform micro-aeration.

In an embodiment, the anaerobic digestion reactor is an anaerobic sequencing batch reactor, and the electrolytic reactor is a column air-lift electrolytic reactor.

### BRIEF DESCRIPTION OF DRAWINGS

The above and other objects and advantages of this invention may be more clearly seen when viewed in conjunction with the accompanying drawing wherein:

FIG. 1 depicts a flow diagram of a poultry litter digester constructed in accordance with an exemplary embodiment.

FIG. 2 depicts a schematic diagram of an anaerobic digestion reactor constructed as an anaerobic sequencing batch reactor in accordance with an exemplary embodiment.

FIG. 3 depicts a schematic diagram of the anaerobic digestion reactor constructed as a semi-continuous stirred tank reactor in accordance with another exemplary embodiment, where HL1 and HL2 reference two high rings on the float meter rod, LL1 and LL2 reference two low rings on the float meter rod, NF references a normally off port on relay switch, and NO references a normally on port on relay switch.

FIG. 4 depicts a schematic diagram of an electrolytic reactor constructed as a column-shaped airlift electrolytic crystallizer in accordance with an exemplary embodiment.

FIG. 5 depicts a schematic diagram of an electrolytic reactor constructed as a dual-chamber electrolytic struvite precipitator in accordance with an exemplary embodiment.

FIG. 6 depicts a process for processing poultry litter in accordance with an exemplary embodiment.

FIG. 7A depicts the variation of daily biogas production (L) and effluent COD (mg O₂/L) from the anaerobic co-digestion of poultry litter with wheat straw in Reactor 1 of Example 1.

FIG. 7B depicts the variation of daily biogas production (L) and effluent COD (mg O₂/L) from the anaerobic co-digestion of poultry litter with wheat straw in Reactor 2 of Example 1.

FIGS. 8A through 8D depict the effect of up-flow velocity on particle size distribution of struvite precipitates recovered from simulated poultry wastewater (current density j=12.4 A/m², HRT=4 hours) in Example 2.

FIG. 9 depicts PO₄³⁻, NH₃—N, and COD removal efficiency after 4 hours of treatment at constant current with no pH control (j=12.4 A/m², U_{sg}=107.42 m/h) in Example 2.

FIG. 10A depicts pseudo-first-order kinetics of NH₃—N recovery through struvite precipitation using the airlift electrolytic reactor of Example 2.

FIG. 10B depicts pseudo-first-order kinetics of PO₄³⁻ recovery through struvite precipitation using the airlift electrolytic reactor of Example 2.

FIG. 11 depicts the evolution of pH and electric conductivity during treatment of ADPW (U_{sg}=107.42 m/h, j=12.4 A/m²) in Example 2.

FIG. 12 depicts the calculated saturation indices for expected precipitates at pH 7.8 to 9.22, j=12.4 A/m², temperature=25° C. in Example 2.

FIG. 13A depicts FT-IR of the recovered precipitates from ADPW in Example 2.

FIG. 13B depicts SEM-EDS of the recovered precipitates from ADPW in Example 2.

FIG. 13C depicts XRD profiles of the recovered precipitates from ADPW in Example 2.

FIG. 13D depicts PSD (lognormal fitting) of the recovered precipitates from ADPW in Example 2.

FIG. 14 depicts a schematic diagram of a batch anaerobic digestion reactor for micro-aeration tests in Example 4.

### DETAILED DESCRIPTION

While this invention is susceptible to embodiment in many different forms, there are shown in the drawings and will herein be described hereinafter in detail some specific embodiments of the invention. It should be understood, however, that the present disclosure is to be considered an exemplification of the principles of the invention and is not intended to limit the invention to the specific embodiments so described.

A poultry litter digester 100 is disclosed for converting poultry litter into a slow-release phosphorous fertilizer (e.g., magnesium ammonium phosphate (MgNH₄PO₄·6H₂O), also known as “struvite” and biogas. As used herein, the term “poultry litter” includes chicken feces and may also include bedding material such as straw, sawdust, wood shavings, shredded paper, and peanut or rice hulls.

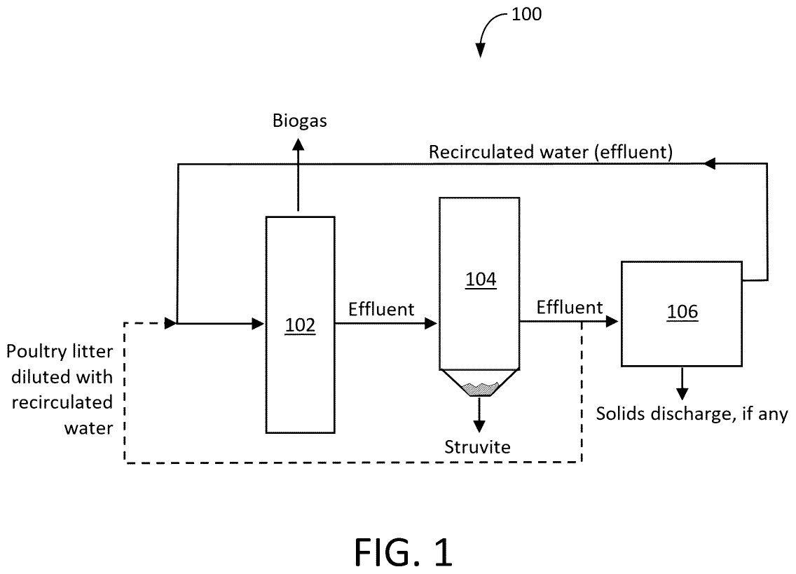

In one embodiment, as depicted in FIG. 1, the poultry litter digester 100 includes an anaerobic digestion reactor 102, an electrolytic reactor 104 downstream from the anaerobic digestion reactor 102, and a water reclamation system 106 downstream from the electrolytic reactor 104. This poultry litter digester 100 maintains throughput treatment capacity while reducing water loss for recycling. It will be appreciated that the water reclamation system 106 is an optional component and that other embodiments of the poultry litter digester 100 may not incorporate this unit if higher quality effluent is obtained from the electrolytic reactor 104. The dotted line in FIG. 1 indicates an exemplary water return route from the electrolytic reactor 104 directly to the anaerobic digestion reactor 102 where the water reclamation system 106 is omitted.

The anaerobic digestion reactor 102 digests poultry litter with one or more external carbon-rich streams, such as agricultural straw waste, to achieve a balanced carbon-to-nitrogen ratio (C/N). By employing anaerobic co-digestion, the anaerobic digestion reactor 102 solves several common problems associated with traditional anaerobic digestion. For example, anaerobic co-digestion offers enhanced digestibility, improved process stability, and a better digestate product with a higher nutrient value versus traditional designs.

Depending on the operating conditions, various anaerobic digestion reactors 102 will be suitable for use in the poultry litter digester 100, including but not limited to anaerobic sequencing batch reactors (ASBR), plug flow reactors (PFR), induced blanket reactors (IBR), upflow anaerobic sludge blankets (UASB), continuous stirred tank reactors (CSTR), and anaerobic filters (AF). The anaerobic digestion reactor 102 may facilitate anaerobic digestion using micro-aeration, i.e., dosing with small quantities of air or oxygen. In various embodiments, air or oxygen is introduced to the anaerobic digestion reactor 102 via a single injection, intermittently (pulse-mode), or continuously. Dosing can also be carried out at different stages of the digestion process (pretreatment, during digestion, or post digestion).

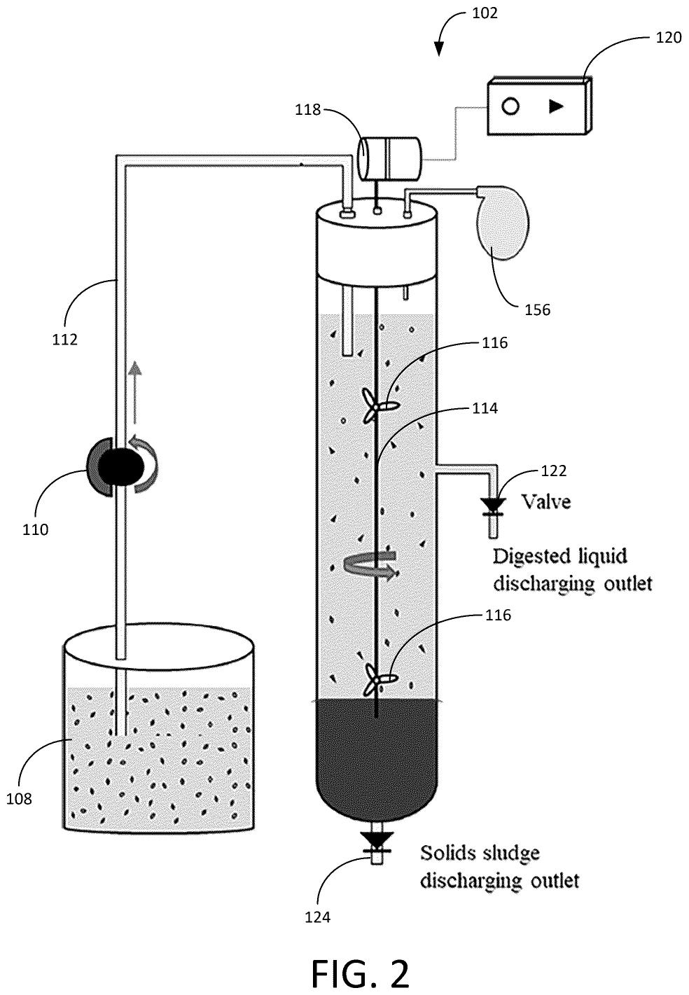

In one embodiment, as depicted in FIG. 2, the anaerobic digestion reactor 102 is an advanced liquid ASBR system (e.g., an 18 L cylindrical reactor with a full working volume of 16 L and 2 L headspace for maintaining enough biogas partial pressure). To prepare the feedstock for anaerobic co-digestion in the anaerobic digestion reactor 102, poultry litter and a carbon-rice source (e.g., straw waste) are pretreated (e.g., milled) to make particles sufficiently small and uniform in size for the treatment, then diluted with water to obtain a substrate solution. The poultry litter and the carbon-rich source are optionally sieved prior to dilution to screen out particles that are too large. The resulting substrate solution is fed from a feeding tank 108 to the anaerobic digestion reactor 102, e.g., using a peristaltic pump 110 and feeding tubes 112, at a predetermined organic loading rate (OLR, g VS/L_{reactor}/day d, where VS is the volatile solids content in the substrate and L is the reactor volume). The substrate solution may be mixed in the anaerobic digestion reactor 102 using a stirring rod 114 with a propeller 116, which is optionally connected with an agitator 118 and a speed controller 120 to control the mixing feed. Inoculum sludge (i.e., a liquid sludge used as the microbial inoculum) is also added into the anaerobic digestion reactor 102. To initiate anaerobic co-digestion, the OLR is gradually increased (e.g., by 0.4 g VS/L/d every two days until day 16) to reach an appropriate operating level, and then a constant OLR may be used until the steady state of the anaerobic co-digestion process is reached. At steady state, the anaerobic co-digestion may be maintained on an ongoing basis, with intermittent mixing of the substrate solution as subsequent quantities are introduced. Sludge is discharged as necessary to maintain a suitable operating volume (e.g., 4 L) within the anaerobic digestion reactor 102. As shown in FIG. 2, two discharging ports 122, 124—one in the middle and one at the bottom of the anaerobic digestion reactor 102—allow for removal of supernatant (effluent) and sludge, respectively.

The anaerobic digestion reactor 102 is optionally wrapped with a temperature-control element, such as clear vinyl tubing running temperature-controlled water, to maintain a desirable anaerobic co-digestion temperature (e.g., 37±2° C.), which is optionally measured by a digital thermometer. In one embodiment, the water for the temperature-control element is heated in a tank using a bucket heater and fed to the tubing by a submersible water pump.

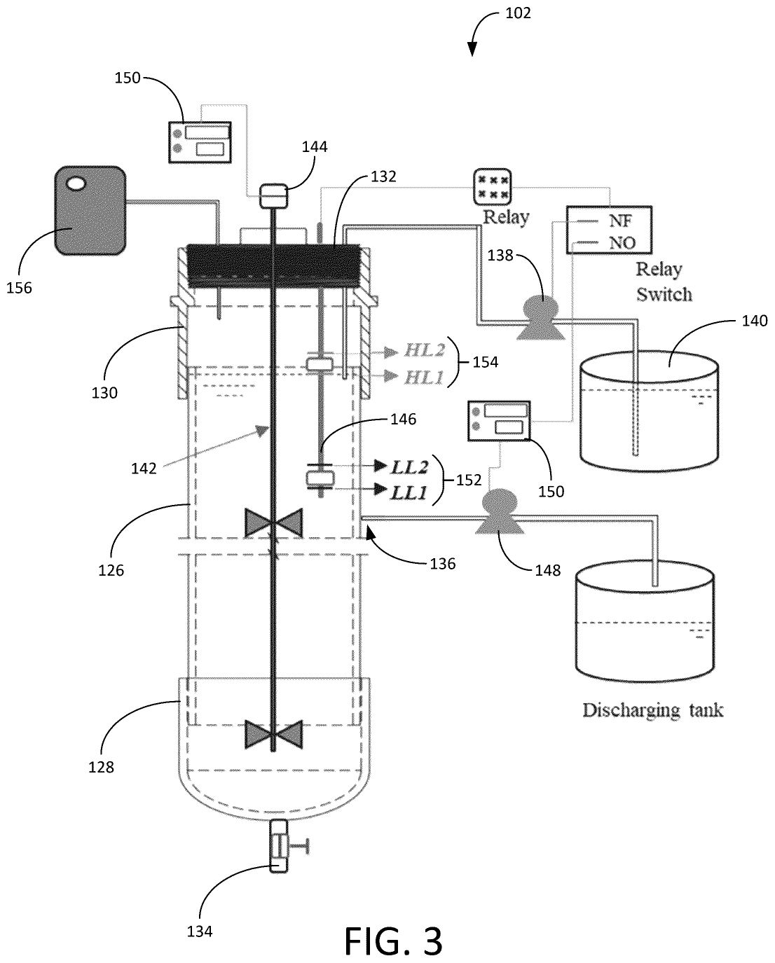

In the embodiment depicted in FIG. 3, the anaerobic digestion reactor 102 is instead a semi-CSTR system having a body 126, a bottom cap 128 attached to the bottom of the body 126, an adapter 130 placed on top of the body 126, and a top cap 132. A solids discharge outlet 134 is configured at the bottom of the anaerobic digestion reactor 102 for discharging solids and a liquids discharge outlet 136 is installed at the side of the body for discharging liquids. The substate solution is fed by a feeding pump 138 from a feeding tank 140 through a feeding outlet at the top of the anaerobic digestion reactor 102. Both liquid feeding and liquid discharge may be controlled with peristaltic pumps. In one embodiment, a stirring rod 142 for a speed-controlled mixer 144 and a liquid level sensor (float meter) 146 are disposed within the body. A peristaltic pump 148 for the liquids discharge outlet 136 is activated by a time relay 150 and stopped when a low-level signal from low rings 152 (LL1, LL2) on the float meter 146 triggers another relay 150 to switch off. Substrate feeding at the feeding outlet is activated when liquid discharge is complete (i.e., upon the low-level signal from the float meter 146) and ended by a high-level signal from the high rings 154 (HL1, HL2) on the float meter 146.

Methane-containing biogas produced from the anaerobic co-digestion process is optionally collected through an outlet in the anaerobic digestion reactor 102, e.g., within a gas bag 156 and/or through a gas collection tube. This biogas produced can be used as heating and cooking fuel, supporting a poultry producer's own energy demands or producing a potential revenue stream. In producing biogas, the anaerobic digestion reactor 102 facilitates the breakdown of organic substances and turns magnesium and phosphorous into an effluent that is available for downstream processing by the electrolytic reactor 104. This breakdown is critical because magnesium and phosphorus in indigested wastes are largely bound to solid organic materials and are not available for further processing.

After anaerobic co-digestion, the resulting anaerobic digested poultry wastewater (ADPW) effluent is characterized by high levels of nutrients (nitrogen and phosphorus) and, in general, by increased ion concentrations of Mg²⁺, NH₄⁺, and PO₄³⁻. Once biogas is captured, the effluent is routed from the anaerobic digestion reactor 102 to the electrolytic reactor 104, which is configured to recover phosphate (PO₄³⁻) and ammonia-nitrogen (NH₃—N) through electrochemical struvite precipitation. As previously noted, struvite precipitation presents a valuable opportunity to reclaim a fertilizer from poultry waste.

The electrolytic reactor 104 employs soluble magnesium electrodes 158, 160 to provide the Mg²⁺ ions needed to support struvite precipitation. More particularly, the electrolytic reactor 104 includes one or more lightly electrically charged magnesium plates 158, 160 that produce Mg²⁺ at anode 158 in electrolysis. The magnesium sacrificial anode 158 facilitates struvite precipitation without the need to add magnesium salts and NaOH to the effluent. The use of an electrolytic reactor 104 also prevents water electrolysis, so no hydrogen gas is produced. Mg²⁺ release may be controlled by adjusting the current density applied. In one embodiment, controlled Mg²⁺ release from the anodic magnesium plate 158 in the electrolytic process is automated. It will be appreciated that the magnesium consumption required from the electrolytic reactor 104 can be reduced where the effluent received from the anaerobic digestion reactor 102 already has increased Mg²⁺ content.

When the magnesium plates 158, 160 produce Mg²⁺ at anode in electrolysis, OH⁻ radicals are simultaneously generated at the cathode, which can increase the liquid pH. In this way, the two requirements for struvite formation, i.e., sufficient Mg²⁺ concentration and a raised pH in the liquid, can be easily met. The electrolytic reactor 104 separates the nitrogen and phosphorous nutrients from the effluent and drops the same into a settling tank as struvite, which is subsequently dried to form a powder. Depending on the amount of phosphate and ammonium present, approximately 1 gram of struvite precipitates from each one liter of effluent.

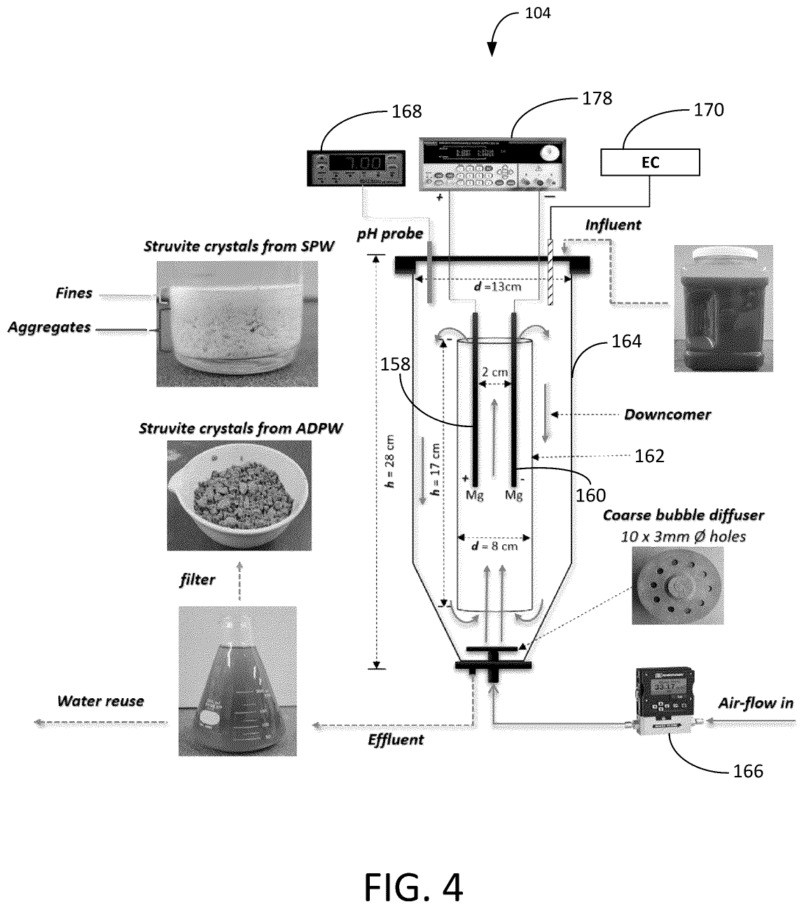

In one embodiment, as shown in FIG. 4, the electrolytic reactor 104 is a column air-lift electrolytic reactor (ALER) having two concentric tubes for struvite recovery, namely an internal draft tube (riser) 162 and an external column 164 having a wider diameter than the internal draft tube 162. During operation, gas bubbles from a sparger in the electrolytic reactor 104 move upward into the draft tube 162 as compressed air is introduced at the bottom of the electrolytic reactor 104 using a mass flow controller 166, which enables liquid circulation flow between the riser and the downcomer to ensure adequate mixing inside the electrolytic reactor 104. In one embodiment, the pH and conductivity of effluent within the electrolytic reactor 104 are monitored using a digital pH controller 168 and a conductivity meter 170, respectively.

The depicted embodiment from FIG. 4 accomplishes the following non-limiting design objectives: (i) it provides sufficient mixing to promote interactions between struvite crystals to form larger crystals with a narrow size distribution that are easy to settle and harvest; (ii) it maintains a high throughput capacity without sacrificing the treatment efficiency and quality of the struvite product; (iii) struvite product harvesting from the depicted electrolytic reactor 104 is not cumbersome, and the liquid loss due to struvite removal is minimal; and (iv) the depicted electrolytic reactor 104 is suitable for scale-up operations and can be conveniently tailored to accommodate different commercial applications.

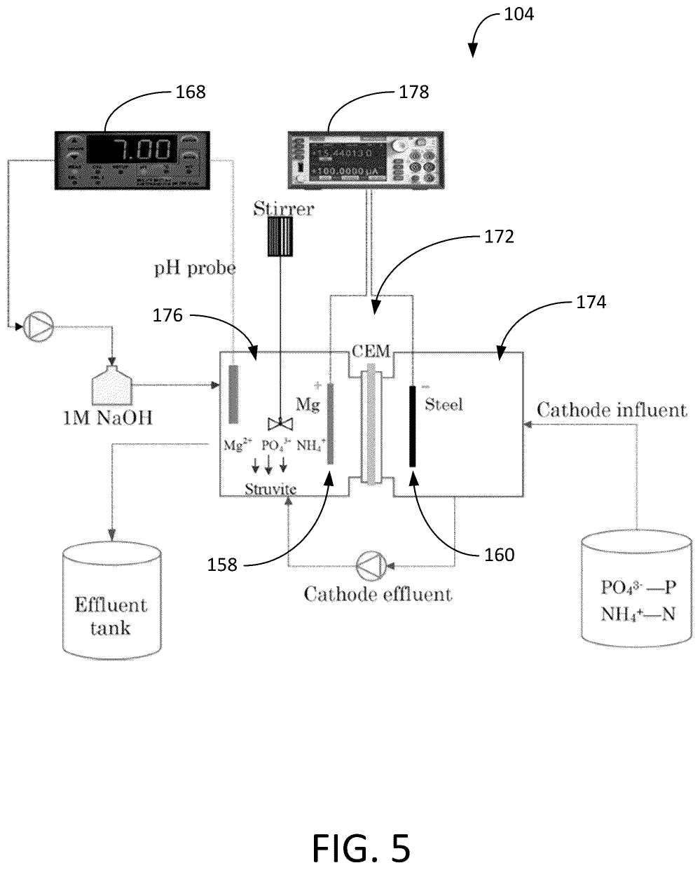

Turning to another embodiment, as shown in FIG. 5, the electrolytic reactor 104 is a dual-chamber electrolytic struvite precipitator in which a cation exchange membrane 172 is used to separate the cathode chamber 174 from the anode chamber 176 to prevent the migration of anions from anode to cathode and to induce a bulk phase pH increase in the cathode chamber 174. Electrodes are placed symmetrically on one side of the membrane 172, and a constant voltage is applied across the electrodes using a DC power supply 178. At the beginning of electrolysis, both the cathode and anode chambers 174, 176 are filled with the effluent, and a small amount of current is applied to the system using the power supply 178. After the pH in the cathode chamber 174 is raised to the desired level, the anode chamber 176 is drained and replaced with effluent from the cathode chamber 174, which is then refilled with a new batch of effluent. This operation is repeated to achieve cyclic treatment with struvite precipitation in the anode chamber 176.

The ALER system creates larger, more uniform struvite crystals than the dual-chamber reactor. More particularly, the ALER lacks mechanical contacts and, therefore, can suppress or reduce the secondary nucleation rate to grow crystals of large sizes. The ALER also provides a high surface-to-volume ratio which allows adequate mixing of reactants with a low shear force and high liquid-solid mass transfer rate due to increased liquid circulation rates. Further, the circulation flow within the ALER can keep struvite crystals suspended for a long time, allowing them to grow. The gas phase can also help with pH regulation by stripping CO₂ from the solution, resulting in a slight pH increase which helps to resist pH drops caused by struvite formation. As a result, significantly less chemicals are needed to maintain the pH at the desired operating value. Column-shaped ALERs are also easy to scale up, and foam production between electrodes, which is often a significant problem when treating organic-rich wastewater in electrochemical reactors, can be minimized by controlling the up-flow velocity.

After processing, the precipitated struvite is collected in a settling tank (not shown) or within the electrolytic reactor 104, and the remaining effluent is removed. Although most of the nutrients are precipitated by the electrolytic reactor 104 and between 80% and 90% of the water from the effluent comes out from the electrolytic reactor 104 clear, a small amount of “rejected” water is created that also contains a small amount of nutrients. In some embodiments, the rejected water is disposed of or is reused in a separate context, e.g., as a road treatment for ice and snow prevention in the wintertime, without further processing at the poultry litter digester 100. In another embodiment, the poultry litter digester 100 recovers the rejected water and recycles it to a reclaimed water for reuse. The rejected water must be cleaned enough to dilute incoming poultry litter. To accomplish this recycling, the rejected water is routed to a water reclamation system 106 for further processing after struvite precipitation. The water reclamation system 106 performs water clarification on the rejected water. In one embodiment, the water reclamation system 106 is a forward osmosis reactor that is configured to remove the majority of minerals and other ions that are not removed by the electrolytic reactor 104. The forward osmosis reactor filters the rejected water through a semipermeable membrane and uses the natural energy of osmotic pressure to separate liquid from the solids in the solution. It will be appreciated that different membranes may be used for the forward osmosis reactor.

Once recycled, the reclaimed water can be recirculated to the anaerobic digestion reactor 102 to liquefy dry poultry litter for continuous digestion, thus minimizing the water input required for anaerobic co-digestion. In one embodiment, peristaltic pumps are used to transfer the reclaimed water back into the anaerobic digestion reactor 102. After recycling for some time, the rejected water has a reduced volume and minimal N and P content and can no longer be recycled to the anaerobic digestion reactor 102. This final rejected water is removed from the poultry litter digestion reactor and can be land applied locally without posing environmental pollution risks.

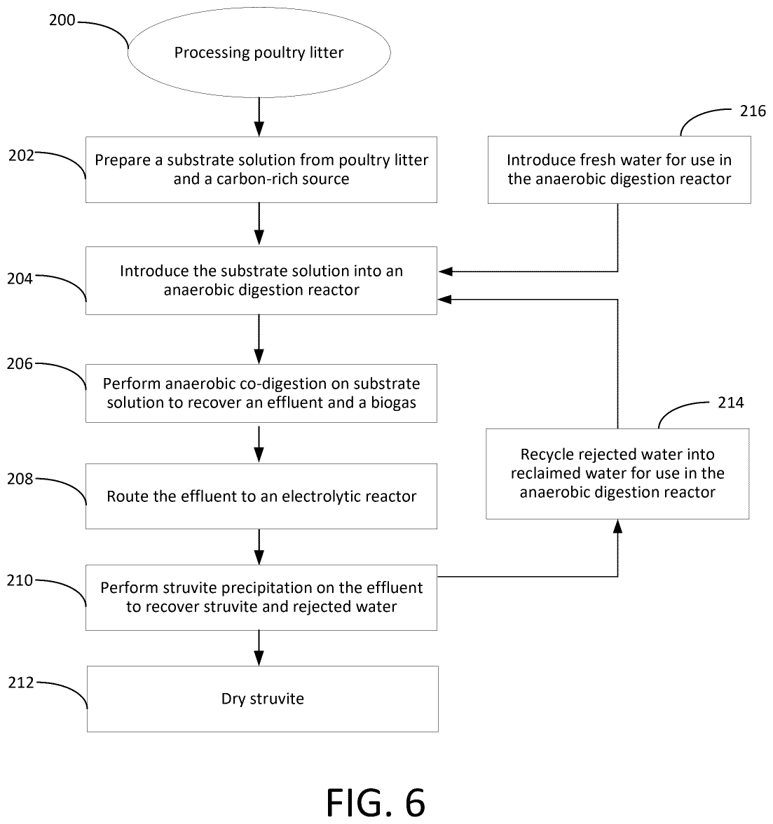

Turning to FIG. 6, in one aspect, a process 200 is disclosed for processing poultry litter. The process includes step 202 of preparing a substrate solution from poultry litter and a carbon-rich source, step 204 of introducing the substrate solution into an anaerobic digestion reactor 102, and step 206 of performing anaerobic co-digestion on the substrate solution within the anaerobic digestion reactor 102 to recover an effluent and a biogas. After step 206, step 208 entails route the effluent to an electrolytic reactor 104, where struvite precipitation is performed at step 210 to recover struvite (dried at step 212) and rejected water. Optionally, the process includes a step 214 of recycling rejected water into a reclaimed water for use in the anaerobic digestion reactor 102. On each cycle, step 214 may be performed instead of or in addition to step 216 of introducing fresh water for use in the anaerobic digestion reactor 102, and vice versa.

### EXAMPLES

The process and system for poultry litter digestion is further illustrated by the following Examples, which are provided for the purpose of demonstration rather than limitation.

### Example 1—Set Up

This Example was designed to evaluate anaerobic co-digestion of poultry litter with wheat straw in a daily-schedule-based operated ASBR system. Two identical anaerobic digestion reactors with a working volume of 16 L were built and set up for experimental runs. The reactor body was covered with hot water circulation and cotton insulation to maintain the temperature (35° C.±2° C.). The reactor was operated in an ASBR mode with cycle time maintained at 24 h (one day). A full operation cycle included an effluent discharging period after the solids settling, an immediately followed substrate feeding period, and two solids settling (reaction) periods divided by two mixing periods (each for 10 min).

For the substrate, raw poultry litter and wheat straw were collected from a poultry farm and a grass farm, respectively. The poultry litter had a total carbon (TC) of 27.20±2.92%, total nitrogen (TN) of 3.37±0.65%, and a moisture content of 21.97±0.31%, while the wheat straw had a relatively higher TC of 43.47±0.15%, and a relatively lower TN of 0.76±0.03%, and a relatively lower moisture content of 9.23±0.11%. The desired C/N ratio and TS level could be reached by balancing the weight proportions of poultry litter, wheat straw, and water. Two types of inoculum sludge were collected from two local wastewater treatment plants.

The three key operation parameters including substrate C/N ratio, substrate TS (%), and HRT (d) were examined, each at five levels including±alpha (α=1.682), ±1 (high and low), and 0 (medium) as the coded values. The actual values of the high and low levels of each factor were 25 and 15, 8 and 4, and 21 and 11 for the C/N ratio, TS, and HRT, respectively. A total of 20 experimental runs were generated, including 6 replicates of the center values (0, 0, 0), 8 cube points (±1, ±1, ±1), and 6 axial points (0, 0, ±1.682; 0, ±1.682, 0; and ±1.682, 0, 0). Even with the reduced experimental points, CCD could still accurately determine the contour behavior of the fitting model. The coded values and the corresponding actual values of the levels of each variable in different runs in the two ASBR systems were described in Tables 1 and 2.

TABLE 1
Reactor 1.

| | | | | | | | | | Methane production (mean | |
|---|---|---|---|---|---|---|---|---|---|---|
| | | | | | | | | | values and standard | |
| | | | | | | | | | deviation of biogas | |
| | | | | | | | Substrate | | production rate and | |
| | | | | | | | composition | | methane production rate | |
| | Input variables (Code value) | | | | | | based on | | were provided based on 3- | |
| | C/N | | | | HRT | | fresh | | 11 days' measurements) | |
| | ratio | | TS (%) | | (d) | | weight (%, | OLR (g | BPR | MPR (L |
| Run | (X1) | | (X2) | | (X3) | | PL:WS) | VS/LR/d) | (L/LR/d) | CH4/LR/d) |
| 1 | 20 | (0) | 6 | (0) | 16 | (0) | 2.90:4.11 | 2.47 | 0.60 ± | 0.30 ± 0.03 |
| | | | | | | | | | 0.05 | |
| 2 | 20 | (0) | 6 | (0) | 24.4 | (1.68) | 2.91:4.13 | 1.61 | 0.39 ± | 0.20 ± 0.02 |
| | | | | | | | | | 0.03 | |
| 3 | 15 | (−1) | 4 | (−1) | 21 | (1) | 2.79:2.02 | 1.19 | 0.29 ± | 0.14 ± 0.02 |
| | | | | | | | | | 0.02 | |
| 4 | 15 | (−1) | 4 | (−1) | 11 | (−1) | 2.78:2.02 | 2.28 | 0.70 ± | 0.37 ± 0.03 |
| | | | | | | | | | 0.06 | |
| 5 | 20 | (0) | 6 | (0) | 7.6 | (−1.68) | 2.90:4.12 | 5.20 | 1.18 ± | 0.72 ± 0.09 |
| | | | | | | | | | 0.14 | |
| 6 | 15 | (−1) | 8 | (1) | 21 | (1) | 5.57:4.04 | 2.33 | 0.46 ± | 0.21 ± 0.02 |
| | | | | | | | | | 0.02 | |
| 7 | 25 | (1) | 4 | (−1) | 11 | (−1) | 1.39:3.22 | 2.53 | 0.70 ± | 0.4 ± 0.04 |
| | | | | | | | | | 0.07 | |
| 8 | 25 | (1) | 8 | (1) | 21 | (1) | 2.78:6.45 | 2.67 | 0.57 ± | 0.26 ± 0.03 |
| | | | | | | | | | 0.04 | |
| 9 | 20 | (0) | 6 | (0) | 16 | (0) | 2.90:4.11 | 2.47 | 0.61 ± | 0.30 ± 0.01 |
| | | | | | | | | | 0.02 | |
| 10 | 11.6 | (−1.68) | 6 | (0) | 16 | (0) | 5.50:1.88 | 2.16 | 0.41 ± | 0.19 ± 0.02 |
| | | | | | | | | | 0.03 | |
| 11 | 20 | (0) | 2.6 | (−1.68) | 16 | (0) | 1.28:1.81 | 0.94 | 0.28 ± | 0.16 ± 0.02 |
| | | | | | | | | | 0.01 | |
| 12 | 20 | (0) | 6 | (0) | 16 | (0) | 2.90:4.11 | 2.47 | 0.56 ± | 0.29 ± 0.01 |
| | | | | | | | | | 0.03 | |

TABLE 2
Reactor 2.

| | | | | | | | | | Methane production (mean | |
|---|---|---|---|---|---|---|---|---|---|---|
| | | | | | | | | | values and standard | |
| | | | | | | | | | deviation of biogas | |
| | | | | | | | Substrate | | production rate and | |
| | | | | | | | composition | | methane production rate | |
| | Input variables (Code value) | | | | | | based on | | were provided based on 3- | |
| | C/N | | | | HRT | | fresh | | 11 days' measurements) | |
| | ratio | | TS (%) | | (d) | | weight (%, | OLR (g | BPR | MPR (L |
| Run | (X1) | | (X2) | | (X3) | | PL:WS) | VS/LR/d) | (L/LR/d) | CH4/LR/d) |
| 1 | 20 | (0) | 6 | (0) | 16 | (0) | 2.90:4.11 | 2.47 | 0.61 ± | 0.31 ± 0.02 |
| | | | | | | | | | 0.05 | |
| 2 | 15 | (−1) | 8 | (1) | 11 | (−1) | 5.56:4.04 | 4.46 | 0.88 ± | 0.45 ± 0.05 |
| | | | | | | | | | 0.07 | |
| 3 | 25 | (1) | 4 | (−1) | 21 | (1) | 1.39:3.22 | 1.32 | 0.38 ± | 0.21 ± 0.02 |
| | | | | | | | | | 0.03 | |
| 4 | 25 | (1) | 8 | (1) | 11 | (−1) | 2.77:6.44 | 5.10 | 0.86 ± | 0.43 ± 0.08 |
| | | | | | | | | | 0.15 | |
| 5 | 28.4 | (1.68) | 6 | (0) | 16 | (0) | 1.66:5.18 | 2.71 | 0.60 ± | 0.3 ± 0.01 |
| | | | | | | | | | 0.02 | |
| 6 | 20 | (0) | 6 | (0) | 16 | (0) | 2.90:4.11 | 2.47 | 0.65 ± | 0.33 ± 0.01 |
| | | | | | | | | | 0.02 | |
| 7 | 20 | (0) | 9.4 | (1.68) | 16 | (0) | 4.53:6.42 | 3.77 | 0.63 ± | 0.29 ± 0.06 |
| | | | | | | | | | 0.12 | |
| 8 | 20 | (0) | 6 | (0) | 16 | (0) | 2.90:4.11 | 2.47 | 0.56 ± | 0.28 ± 0.01 |
| | | | | | | | | | 0.03 | |

The selected inoculum sludge was analyzed in physio-chemical properties and was used to start up the anaerobic co-digestion process. Twenty (20) experimental runs were performed, and the feeding substrate compositions for each experimental run were calculated (see Table 1). The center values of the operation parameters were employed in the first 3-6 days of operation as the adaptation period in each experimental run and were also adopted when inhibitions occurred.

The TC and TN of the poultry litter and wheat straw were measured by combustion using Elementar varioMax CN Cube (Elementar, German). TS, moisture content, and volatile solids (VS) were analyzed by thermogravimetric analysis. For the effluent liquid samples, the concentration of chemical oxygen demand (COD), TN, ammonium nitrogen (NH₄⁺—N), nitrite nitrogen (NO₂⁻—N), nitrate nitrogen (NO₃⁻—N), phosphorus, and magnesium (Mg²⁺) were analyzed using a DR 3900 Hach Spectrophotometer and Hach vials (Hach Company, IA, US). All the data was measured in triplicates and mean values with standard deviations were obtained.

For the biogas analysis, the volumes of daily collected biogas were measured using a wet gas test meter (Model XMF-1, Shanghai Cixi Instrument Co., ltd, Chin). The compositions of biogas samples were analyzed using a Shimadzu Gas Chromatograph (GC 2014, Shimadzu Scientific Instruments, Inc., Maryland, CO, USA) equipped with a thermal conductivity detector (TCD).

The microbial community was analyzed for inoculum selection. Microbial community analysis DNA extractions were performed using the DNeasy PowerLyzer PowerSoil Kit (Qiagen, Germantown, MD). The raw reads were submitted to the NCBI (National Center for Biotechnology Information) Sequence Read Archive (SRA). The microbial inoculum was selected based on microbial community analysis.

### Example 1—Results

At the phylum level, inoculum (a) mainly contained the phylum of Bacteroidota (44%), followed by Firmicutes (22%) and Chloroflexi (13%). While in inoculum (b), the most abundant phylum was Chloroflexi (42%), followed by Actinobacteriota (11%), Cloacimonadota (11%), and Bacteroidota (10%). Compared to inoculum (a), inoculum (b) showed a more diverse microbial structure. In addition, the major phyla in inoculum (b) were found to be likely ubiquitous in most stable anaerobic digestion reactors. Chloroflexi is found responsible for the hydrolysis of both monosaccharides and polysaccharides as well as the production of acetic acid. Actinobacteriota is hydrolytic bacteria that can also decompose polysaccharides and proteins in AD systems. Cloacimonadota is found to be especially important in lipid-rich waste AD. At the genus level, Bacteroides dominated inoculum (a) with an abundance (31%), far exceeding the other genus (lower than 5%). However, the distribution of microorganisms in inoculum (b) was more even. SBR1031 and Longilinea were the two dominant genera (accounting for 16% and 13%, respectively). The abundance of methanogens genus was negligible (lower than 1%) in inoculum (a) but was 2% (Methanosaeta) in inoculum (b), making inoculum (b) a better choice than inoculum (a) for the anaerobic co-digestion process.

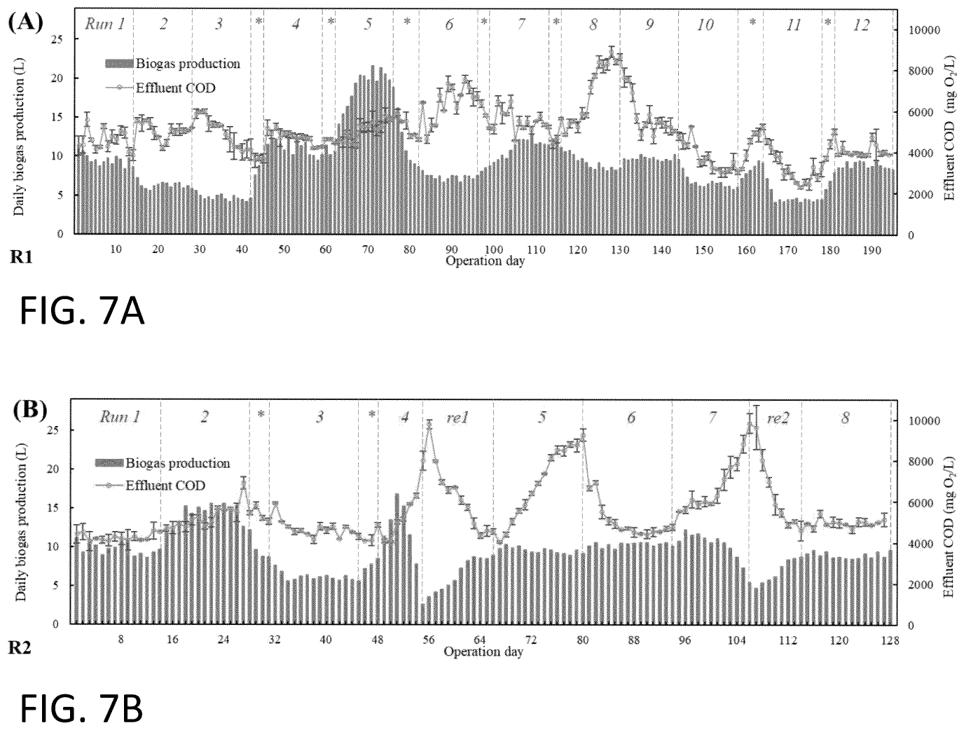

In reactor, the daily biogas production (DBP, L) in each run became stable (difference within 5%) after an adapting period of about 3-5 days (see FIG. 7A), including Run 10 with a C/N ratio of 11.6. The results indicated that the anaerobic co-digestion process in the ASBR could achieve stable and efficient methane production performance under a wide range of operating parameters. The average biogas production rate (BPR, L/LR/d) and the corresponding methane production rate (MPR, L CH₄/LR/d) in the stable stage in each run were calculated and summarized in Table 1. BPR was higher at a lower HRT (Run 5 (7.6 d) compared to the results at center points (16 d) and those in Run 2 (21 d); or Run 4 (11 d) compared to Run 3 (21 d)), indicating that the shorter HRT favored a higher value in BPR. In addition, a higher BPR was found either at the relatively higher C/N ratio or TS level when comparing center points with other runs. A relatively higher C/N ratio or TS level indicated a higher lignocellulosic content in the substrate. As mentioned above, the high abundance of Chloroflexi could be responsible for the efficient hydrolysis of the substrate, leading to a relatively higher methane generation by the methanogens (Methanosaeta). In Reactor 2, however, process inhibitions, where DBP dropped abruptly, were observed in Run 4 and 7. The inhibition in Run 4 might be caused by the combination of a high TS level (8%) and a high C/N ratio coupled with a relatively short HRT (C/N=25, HRT=11 d), which meant a relatively high OLR (5.10 g VS/L/d). While the inhibition in Run 7 should be mainly related to the relatively high TS level (9.36%). However, it was noted that the inhibition was alleviated by adjusting the operating parameters to the center values for a period (of 11 days and 8 days for Reactor 1 and Reactor 2, respectively) as shown in FIG. 7B. The results indicated that the suppression of microorganisms in the ASBR, which lead to inhibition of the anaerobic co-digestion process, could be remedied by implementing appropriate operating parameters.

In summary, the anaerobic co-digestion process in the ASBRs showed continuous methane production with a BPR ranging from 0.28 to 1.18 (L/LR/d). The highest BPR was reached in Run 5 of Reactor 1 with C/N ratio=20, TS level=6%, and HRT=7.6 d, being (1.18±0.14) L/LR/d, where the MPR was (0.72±0.09) L CH4/LR/d. Furthermore, a modified second-order quadratic model (Equation 1) that best fitted the experimental data could be built for BPR using the response surface methodology. Analysis of variance (ANOVA) (see supplementary material) showed that the model was significant (p<0.0001) with a correlation coefficient (R₂) of 0.9724. And it also showed that all the single factors had a significant (p<0.05) effect on the BPR.

$$BPR ( L / {L}_{R} / d ) = 1.11 + 0.03 * C / N + 0.18 * TS - 0.16 * HRT - 0.011 * {TS}^{2} + 0.003 * {HRT}^{2} \qquad ( Equation 1 )$$

Turning to the effects on effluent quality and, more particularly, the effects on COD, in Reactor 1, the effluent COD showed an obvious decreasing trend and tended to become relatively stable at the last 3-4 days (FIG. 7A) in Run 3 (4116.0±509.1 mg O2/L) and Run 11 (3199.5±348.6 mg O2/L), which had the lowest OLRs among all runs. While the runs with the relatively higher OLRs (Run 5 in Reactor 1, Run 2 in Reactor 2) both showed a small increasing trend in the effluent COD, suggesting that a higher OLR might result in a higher effluent COD of the ASBR. This might be related to the development of hydrolytic bacteria under the higher OLR, leading to the enhanced hydrolysis of substrate organics, which also explained the relatively higher BPR as stated previously. Process inhibitions in Run 4 and Run 7 of Reactor 2 with high TS or OLR as mentioned previously could also be evidenced by the rapid increase in the effluent COD as shown in FIG. 7B. COD accumulation was found to be an important reason causing inhibition of the AD process for methane production. At high OLRs in the ASBR, the enhanced hydrolytic bacterial activity resulted in COD or volatile fatty acids accumulation and pH reduction, which caused the suppression of methanogenic microorganisms, ultimately leading to process inhibition. However, it should be noted that the increase in effluent COD was alleviated by the process recovery (re1 and re2) during which the center values of parameters were employed, indicating that appropriate substrate loading can gradually balance microbial activities and alleviate overloading stress, ultimately leading to a stable process.

When evaluating the effects on nitrogen, phosphorus, and magnesium, in the effluent of most runs, the concentrations of different nitrogen species were similar with NH4⁺-N in the range of 120-160 mg/L, NO3⁻—N in the range of 17-34 mg/L, and NO₂⁻—N in the range of 0.7-1.2 mg/L, and TN in the range of 400-500 mg/L (see supplementary material). However, there are some exemptions. In Run 4 of Reactor 1, total organic nitrogen (TON) was lower although NH4+-N was higher (177.3±1.0 mg/L), compared to other runs probably due to its relatively lower C/N ratio (15). In Run 10 of Reactor 1, which had the lowest C/N ratio (11.6), the effluent showed relatively high nitrogen concentrations in different forms including TN (548.0±22.6 mg/L) and NH₄⁺—N (224.6±2.0 mg/L) (see supplementary material), indicating that a low C/N ratio could lead to high nitrogen concentration in the effluent. At a relatively lower C/N ratio, which meant a higher proportion of PL in the mixture substrate, the bacteria activities released relatively more nitrogen, which was mainly from the degradation of protein and lipid in poultry litter, by Actinobacteriota and Cloacimonadota, respectively. The effluent phosphorus concentrations in most runs were stable in the range of 440-500 mg/L (see supplementary material). However, several runs including Run 7 and 8 in Reactor 1 and Run 3 in Reactor 2 (all had a relatively high C/N ratio of 25) showed relatively higher effluent phosphorus (520-650 mg/L). The phosphorus concentration was trending down for Runs 4 and 7 in Reactor 2 with inhibitions. In all, it could be inferred that the effluent phosphorus level could be affected by many factors such as the C/N ratio, OLR, and process stability of the ASBR. The magnesium level in the effluent was generally in the range of 160 to 230 mg/L, except in Run 8 of Reactor 1 and Run 3, 4, and 5 of Reactor 2 which showed a higher range of 260 to 300 mg/L. The release of phosphorus and magnesium was related to the efficiency of bacterial activity that degraded the substrate solids, which was not only determined by the substrate characteristics but also affected by the process stability.

In conclusion, the inoculum with a diverse microbial structure that included 2% of methanogens (Methanosaeta) was chosen for the anaerobic co-digestion of poultry litter with wheat straw in the two ASBRs. The highest BPR (1.18±0.14 L/LR/d) and MPR (0.72±0.09 L CH4/LR/d) were obtained at C/N=20, TS=6%, and HRT=7.6 d. The modified quadratic model was significant (p<0.0001) for predicting BPR. Inhibitions due to COD accumulation were relieved by employing the center values of parameters (C/N=20, TS=6%, HRT=16 d). The concentration of nitrogen, phosphorus, and magnesium in the effluent was affected by the C/N ratio, TS, HRT, and process stability.

### Example 2—Set Up

Anaerobic digested poultry wastewater (ADPW) effluent from Example 1 was stored at 4° C. until use for nutrient recovery, i.e., for struvite (magnesium ammonium phosphate) precipitation using an electrolytic reactor. Table 3 shows the average values of the physicochemical properties of the ADPW samples used in this Example. All values in Table 3 are in mg/L except pH and conductivity.

TABLE 3
Physiochemical properties of ADPW effluent.

| Parameters (mg/L) | Value (mean ± S.D.) |
|---|---|
| pH | 7.78 ± 0.1 |
| Conductivity (mS/m) | 13.4 ± 0.1 |
| sCOD | 5137 ± 0.001 |
| PO₄³⁻ | 1397 ± 0.001 |
| NH₃—N | 941 ± 0.001 |
| Mg²⁺ | 123.8 ± 0.5 |
| K⁺ | 164 ± 0.08 |
| Zn²⁺ | 0.2 ± 0.001 |
| Fe²⁺ | 0.82 ± 0.001 |
| Cr²⁺ | 0.02 ± 0.001 |
| Cu²⁺ | 0.1 ± 0.001 |
| Ca²⁺ | 64.58 ± 0.08 |
| Alkalinity (CaCO₃) | 2540 ± 0.28 |

The first set of experiments was carried out with simulated poultry wastewater to study the effects of different air-flow rates on the amount of nutrient removal and particle size distribution (PSD) of precipitate generated. This was done to establish the optimal mixing characteristics of the electrolytic reactor. The chemical components of ADPW were used to prepare the simulated poultry wastewater matrix by combining deionized water with various analytical-grade salts, including ammonium chloride (NH₄Cl, 99.8%), acetic acid glacial (C₂H₄O₂, 99.85%), sodium phosphate monobasic dihydrate (NaH₂PO₄·2H₂O, 99.0%), calcium chloride (CaCl₂, 97%), glucose (C₆H₁₂O₆, 99.5%) and potassium sulfate (K₂SO₄, 99.0%). The solution was uniformly mixed using a magnetic stirrer, and the pH was adjusted using 1 M NaOH before each experiment. Trace elements (Zn, Cr, Cu, and Fe) with concentrations <0.2 mg/L were not added to the composition of the simulated wastewater because such small concentrations do not have a significant effect on struvite formation, especially when the concentration of PO₄³⁻ and NH₃—N are very high.

An air-lift electrolytic crystallizer was set up according to the schematic and geometric details shown in FIG. 4. The reactor had a total working volume of 6 L and was constructed using transparent polyvinyl chloride. The reaction and particle growth zone comprises two concentric tubes with an inner diameter of 8 cm and 13 cm, respectively. During operation, the gas bubbles from the sparger moved upward into the draft tube as compressed air is introduced at the bottom of the reactor using a mass flow controller (C100L, Sierra Instruments, Monterey, CA), which enables liquid circulation flow between the riser and the downcomer to ensure adequate mixing inside the reactor. The pH and conductivity of the wastewater were monitored using a digital pH controller (Thermo Scientific Alpha pH 190) and a conductivity meter (PC850, Apera Instruments, Columbus, OH), respectively. A high-purity Mg alloy (AZ91) [Al: 8.8 wt. %, Zn: 0.71 wt. %, Mn: 0.19 wt. %. Si: 0.029 wt. %, Ca 0.001. wt. %, Zr: 0.001 wt. %, Mg: balance] was used as a sacrificial anode and cathode material. Both electrodes were rectangular, with a geometric surface area of 403 cm² each. Before each experiment, the electrodes were pretreated to remove metal oxides, which can hinder electron transfer from the electrode surface. After polishing with sandpaper, both electrodes were washed with 1 M HCl solution, rinsed with deionized water, and placed in the draft tube (2 cm apart) to reduce foaming between electrodes and scale accumulation on the anode surface.

To investigate the effect of the up-flow velocity on the PSD of struvite precipitates in the ALER, a total of eight (8) experiments were performed at different flow rates (1, 3, 6, 9 L/min) in duplicates for 4 hrs using simulated poultry wastewater solutions. The air-flow rate was controlled and adjusted using the mass flow controller, and all experiments were conducted in batch mode at room temperature. An applied current (0.5 A) was used with no pH adjustment. The superficial gas velocity (U_{sg}) is the ratio of the gas volumetric flowrate (Q_{g}) to the total cross-sectional area of the inner diameter of the riser (Aᵣ) according to Equation 2:

$${U}_{sg} ( m / h ) = \frac{{Q}_{g} ( L / min ) ( {10}^{- 3} {m}^{3} / L ) ( \frac{60 min}{1 h} )}{{A}_{r} ( {m}^{2} )} \qquad ( Equation 2 )$$

The optimal U_{sg} for mixing was selected and used with the optimum applied current (0.5 A) for batch experiments using the actual ADPW. Experiments with the ADPW were also conducted in duplicate.

Thermodynamic chemical equilibrium was modeled for the relevant analytics. Struvite precipitation from aqueous solutions occurs when Mg, PO₄³⁻, and NH₄⁺ ion concentrations exceed the struvite solubility product, and the process is mainly dependent on the degree of supersaturation inside the reactor. The degree of supersaturation for a system with multiple components is usually expressed in terms of the supersaturation index (SI), which is the logarithmic ratio between the ion activity product [IAP] and solubility product (Kₛₚ). Calculation of the activity coefficients by the model was done using the Davies approximation of the Debye-Huckle equation (see Equations 3 and 4):

$$SI = log \frac{IAP}{{K}_{sp}} \qquad ( Equation 3 )$$

$$IAP = {γ}_{{Mg}^{2 +}} [ {Mg}^{2 +} ] {γ}_{{NH}_{4}^{+}} [ {NH}_{4}^{+} ] {γ}_{{PO}_{4}^{3 -}} [ {PO}_{4}^{3 -} ] \qquad ( Equation 4 )$$

where Kₛₚ=10^{13.26} at 25° C., and γ represents the activity coefficient of the various corresponding struvite-forming species. Its value depends on the valency of the ions and the total ionic strength (I) of the solution, which can be calculated according to the following Equations 5 and 6:

$$log {γ}_{i} = - {AZ}_{i}^{2} 2 i ( [ \frac{\sqrt{1}}{1 + \sqrt{1}} - 0.31 ) \qquad ( Equation 5 )$$

$$1 = 1.6 \times 1 {0}^{- 5} \times E C \qquad ( Equation 6 )$$

where Zᵢ is the valency of species i, EC is electric conductivity in μS/cm, and A is the Debye-Hückel constant with the value of 0.509 at 25° C. All calculations and predictions of mineral speciation and products were performed using the Visual MINTEQ (version 3.1) software package developed by the US Environmental Protection Agency. The chemical species composition of the ADPW sample reported in Table 3 was used as input for the modeling process. The model incorporated pH values from 7.8 to 9.2 in increments of 0.1 units at constant temperature (25° C.).

The efficiency of PO₄³⁻ and NH₃—N recovery as struvite from the ADPW sample was also calculated after each batch experiment using the following Equation 7:

$${N}_{r} ( \% ) = \frac{{[ C ]}_{0} - {[ C ]}_{t}}{{[ C ]}_{0}} \times 100 \% \qquad ( Equation 7 )$$

where Nᵣ is the nutrient (PO₄³⁻ and NH₃—N) removal efficiency; C₀ is the initial concentration of nutrient (mg/L) of the simulated waster before electrolysis; Cₜ is the concentration of nutrient in the anolyte at time, t (mg/L). The purity of the recovered precipitates was determined using SEM-EDS and the acid dissolution method to identify the elemental composition of the crystals. For the acid dilution procedure, 50 mg of recovered solids were dissolved in 50 mL of HCl (0.1 mol/L) and stirred for 10 mins. The solution was then diluted to 100 mL with deionized water and analyzed for NH₃—N content. Assuming that 1 mol of NH₃—N is equivalent to 1 mol of struvite, the purity was calculated according to Equation 8:

$$Productpurity [ \% ] = \frac{{C}_{NH 3 - N} \times {V}_{s}}{{m}_{p}} \times \frac{{M}_{s}}{{M}_{N}} \qquad ( Equation 8 )$$

where C_{NH3—N} is the concentration NH₃—N [mg/L]; Vₛ is the sample volume [0.1 L]; M^{s} is the molecular weight of struvite [245 g/mol]; mₚ is the mass of the precipitate [mg]; and M_{N} is the molecular weight of nitrogen [14 g/mol].

In terms of the reaction kinetics of struvite precipitation, during each experiment, 7 mL samples were collected at 20 min intervals from the reactor and analyzed to determine the changes in COD, PO₄³⁻, and NH₃—N concentrations. PO₄³⁻ was measured using a HACH TNT 843 (0.15-4.5 mg/L) and TNT 846 kits (5-90 mg/L), respectively. NH₃—N was measured using HACH TNT 831 (1-12 mg/L) and HACH TNT 833 (47-130 mg/L). While the COD was analyzed using HACH TNT 823 kit (250-15000 mg/L). All HACH kits were analyzed using a DR 3900 spectrophotometer (Loveland, CO, USA) according to the manufacturer's instructions. The rate of PO₄³⁻ and NH₃—N disappearance or removal from solution over time was evaluated according to a first-order kinetic model as shown in Equations 9:

$$Removalrate ( r ) = - \frac{dC}{dt} = {k}_{i} \times {C}_{i} \qquad ( Equation 9 )$$

$$ln \frac{{[ C ]}_{t}}{{[ C ]}_{0}} = - Kt \qquad ( Equation 10 )$$

where Cₒ and Cₜ are the initial and final concentrations of PO₄³⁻ and NH₃—N (mg/L), respectively, k is the rate constant (min⁻¹), and t is the treatment time (mins). The concentrations of Na, Mg, Ca, K, and heavy metals in the ADPW were quantified by ICP-AES (Optima 5300 DV, Perkin Elmer) before and after treatment. All samples were filtrated by 0.45 μm membrane filters before analysis.

The space-time yield (Y_{ST}) was also calculated to give an overall index of a reactor's performance. As shown in Equation 11, it is the amount of product (mₚ) produced per unit of time (t) and reactor volume (Vᵣ).

$${Y}_{ST} ( Kg / {m}^{3} h ) = \frac{{m}_{p}}{{V}_{r} \times t} \qquad ( Equation 11 )$$

At the end of each experiment, all precipitates in the reactor were collected by vacuum filtration using a glass filter (WHEATON) and air-dried at room temperature for 48 h to characterize the recovered products. After weighing using a balance, the elemental composition of the sample was determined by scanning electron microscope-energy dispersive spectrometer (SEM-EDS) (Zeiss Supra 35, Zeiss, Oberkochen, Germany) analysis. Recovered precipitates were also subjected to X-ray diffraction (XRD) analysis (Siemens D5000, Princeton, NJ, USA) for phase identification and quantification. The functional groups present in the precipitates and the nature of the bonds in the precipitates were analyzed using Fourier transform infrared spectroscopy (FT-IR) (Nicolet iSTM20 Spectrometer, Fisher Scientific, PA, USA).

The PSD of recovered precipitates was obtained by automatic image processing and analysis of crystals using a revolutionary artificial intelligence (AI) technology. For each run, 20 mg of struvite was placed in a vial, and 5 drops of Milli-Q water were added. The vial was then mixed until the crystals were well-dispersed. Three drops of the sample were placed on a glass slide, followed by a coverslip. The specimens were observed under a microscope at ×10 magnification. Images of struvite crystals were captured by an optical microscope (Olympus AX70 Fluorescence Motorized Microscope) connected to a 14 MP HD digital color camera (AmScope MU1403, United Scope, CA, United States). The size distribution (Feret diameter) and statistics were determined using MIPAR image analysis software (Columbus, Ohio, United States) which uses a powerful deep learning technology to effectively recognize particle boundaries and generates size distribution data based on various features of interest. A total of 100 images were captured per sample, and over 5000 crystals were analyzed per sample.

### Example 2—Results

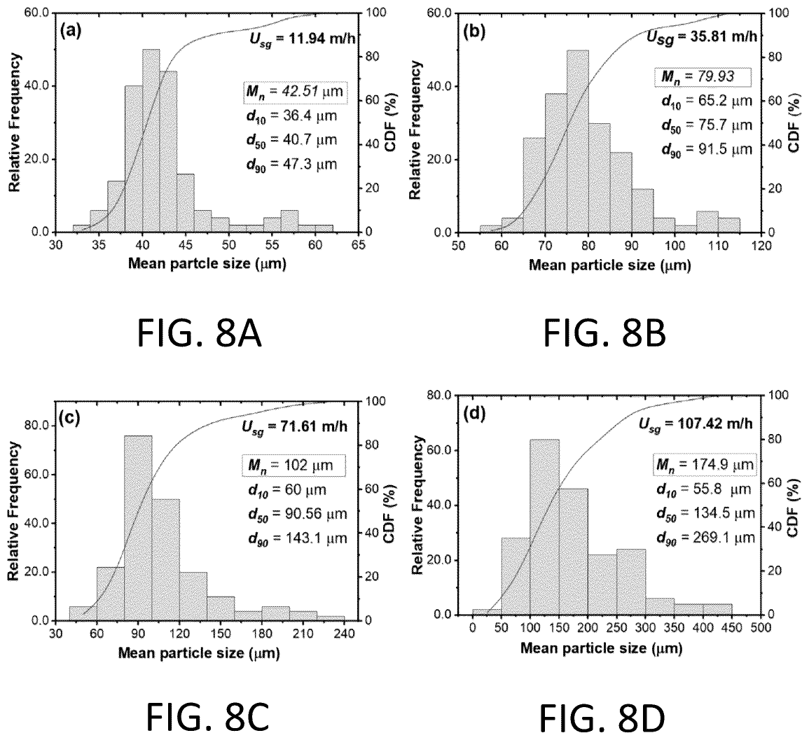

First, the effect of up-flow velocity on particle size distribution was observed during struvite precipitation. The effectiveness of struvite precipitation depends on the crystallizer type and its mode of operation, which is often evaluated from two key aspects: nutrient recovery efficiency and product quality (purity and PSD). In a supersaturated solution, precipitation usually begins with the birth of new particles or nuclei from the liquid phase (nucleation). Once generated, the fate of the nuclei is primarily determined by the thermodynamic and hydrodynamic conditions inside the reaction vessel. Hydrodynamics and mixing promote crystal nucleation and growth by enhancing contact between PO₄³⁻, NH₄⁺, and Mg²⁺ ions; mass transfer of ions from solution to the crystal phase; and settling behavior of generated crystals. Therefore, adequate mixing is essential for effective process operation. In the air-lift crystallizers particles become fully suspended when the up-flow velocity becomes strong enough to counteract the downward pull of gravity. Consequently, only larger particles with a settling velocity equal to or higher than the upward air velocity can settle at the bottom of the reactor. FIGS. 8A through 8D show the effect of different superficial velocities on the PSD of struvite crystals produced by the ALER.

The results indicate that the electrolytic reactor's superficial gas velocity (U_{sg}) is strongly related to the mean particle size of recovered precipitates. The data showed a slight shift of particle size classes to the right, indicating that the mean size of struvite crystals increased with a corresponding increase in U_{sg}. The mean particle size grew from 42.51 μm to 79.93 μm (88.03%) when the U_{sg} was raised from 11.94 m/h to 35.81 m/h. At high U_{sg}, the mean particle size increased from 102 μm at 71.6 m/h to 174.9 μm at 107.42 m/h (71.47%). Overall, the mean particle size increased by 311.43% when the U_{sg} was raised from 11.94 m/h to 107.42 m/h. D50 and D90 values also increased from 40.7 to 134.5 μm and 47.3 to 269.1 μm, respectively, with increasing U_{sg}. At the same time, D10 values changed from 36.4 to 65.2 μm at low U_{sg} (11.94 to 35.81 m/h) and then dropped marginally to 60 and 55.8 μm at 71.61 and 107.42 μm m/h, respectively. However, adjustment of aeration intensity did not improve the nutrient removal efficiency. In general, an increase in U_{sg} will often result in an increase in nucleation rates of struvite crystals due to enhanced molecular mixing between reactants. Additionally, high U_{sg} promotes the suspension of particles and also increases interparticle collisions, leading to the formation of bigger particles (aggregates) with higher sedimentation velocity. However, very high U_{sg} beyond a specific threshold value or optimum will enhance foam formation and cause breakage of crystal aggregates. On the contrary, low U_{sg} and, therefore, slower dissipation of reactants leads to locally higher supersaturation which favors nucleation over crystal growth. This would explain the smaller crystal sizes achieved at low U_{sg}.

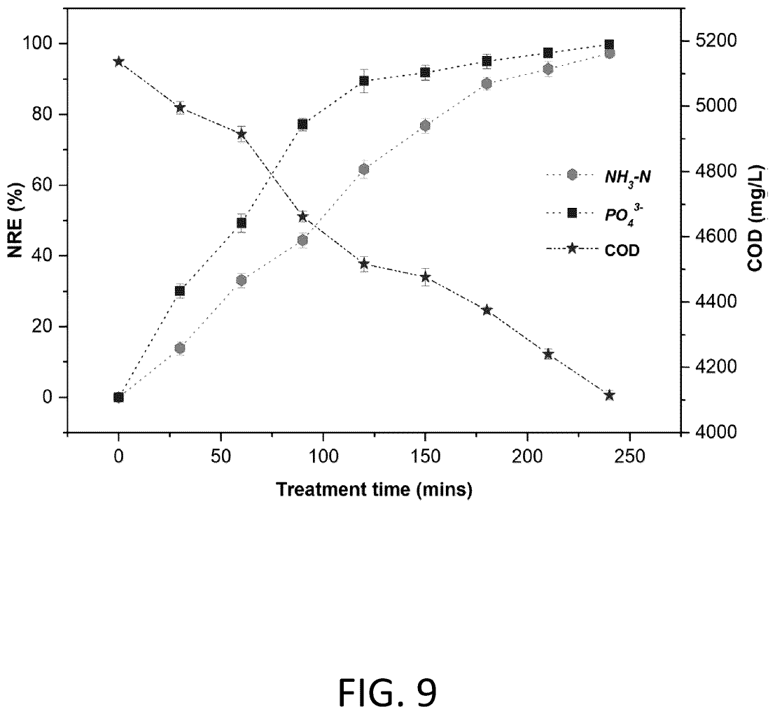

Nutrient recovery efficiency from the ADPW was also investigated. The changes in PO₄³⁻ and NH₃—N removal efficiency and COD concentration changes after 4 hrs of treatment under constant current and air-flow rate are presented in FIG. 9. As expected, PO₄³⁻ and NH₃—N removal efficiencies increased with increasing treatment time. The maximum removal efficiency of PO₄³⁻ and NH₃—N were 99.9% and 97.3%, respectively. At the same time, the COD concentration also decreased slightly from 5137 to 4115 mg/L (19.9%). This decrease in COD levels highlights one advantage of electrochemical systems over conventional chemical struvite precipitation methods. Since most organic compounds often carry a negative charge, COD removal mechanisms in electrochemical systems are usually associated with the following: (a) colloidal particle flocculation by the metal cations (due to Mg²⁺ produced by anodic dissolution), which can neutralize the charge of suspended particles and colloids by the double layer compression, and floc formation, and (b) co-precipitation by metal hydroxide surfaces through complexation or by electrostatic attraction (adsorption).

Predicted metal oxides in the system from chemical equilibrium modeling were Mg(OH)₂, Fe(OH)₂, and Zn(OH)₂. These hydroxides can provide active surfaces for the adsorption of wastewater pollutants and promote flocculation of dispersed particles, which can then be removed during downstream processing steps by filtration or centrifugation. It has also been suggested that foreign substances (colloidal impurities) can facilitate nucleation by reducing the Gibbs free energy needed to produce a crystal nucleus or serve as nuclei for the growth of struvite crystals. The total amount of precipitate recovered at the end of the process was 23.72 g, and the space-time yield was 0.988 kg/m³ h. The efficiency of PO₄³⁻ removal increased steadily from 30.12% after 30 mins to 89.5% after 120 mins of treatment and then almost plateaued. There was little change in PO₄³⁻ removal observed between 150 mins (91.8%) and 240 mins (99.9%) even though the pH was already over 8.9 after 150 mins of treatment. This event was unexpected since it is generally known that at high pH values, the concentration of PO₄³⁻ ions increases, increasing its potential for recovery. Because the ratio of PO₄³⁻:NH₃—N in this Example was 1.5:1, the reduction in PO₄³⁻ removal efficiency after 150 mins of treatment was most likely due to passivation or the formation of a non-conducting oxide on the anode surface which limited electron transfer and corrosion of the Mg alloy. This is a significant issue in electrochemical crystallizers because as the passivation layer thickens, the overpotential required for Mg dissolution increases by several orders of magnitude, thereby limiting the overall efficiency of the process. It is also inconvenient since electrode plates must be manually cleaned, acid-washed, or replaced to remove the protective film on the surface of the AZ91 allow, which takes time. The passive film on Mg alloys usually consists of a chemisorbed oxygen film or compact three-dimensional inner layer of magnesium MgO and a porous outer layer of Mg(OH)₂/MgNH₄PO₄/MgCO₃. MgO is generated directly through the interaction of the AZ91 alloy with the surrounding electrolyte according to the following mechanism in Equation 12:

$$M g + {H}_{2} O \to M g O + 2 {H}^{+} + 2 {e}^{-} \qquad ( Equation 12 )$$

while the production of Mg(OH)₂ is facilitated by the reaction between Mg²⁺ ion and hydroxide ions (OH⁻) generated at the cathode, as shown in Equation 13:

$${Mg}^{2 +} + 2 {OH}^{-} \to {Mg ( OH )}_{2} \qquad ( Equation 13 )$$

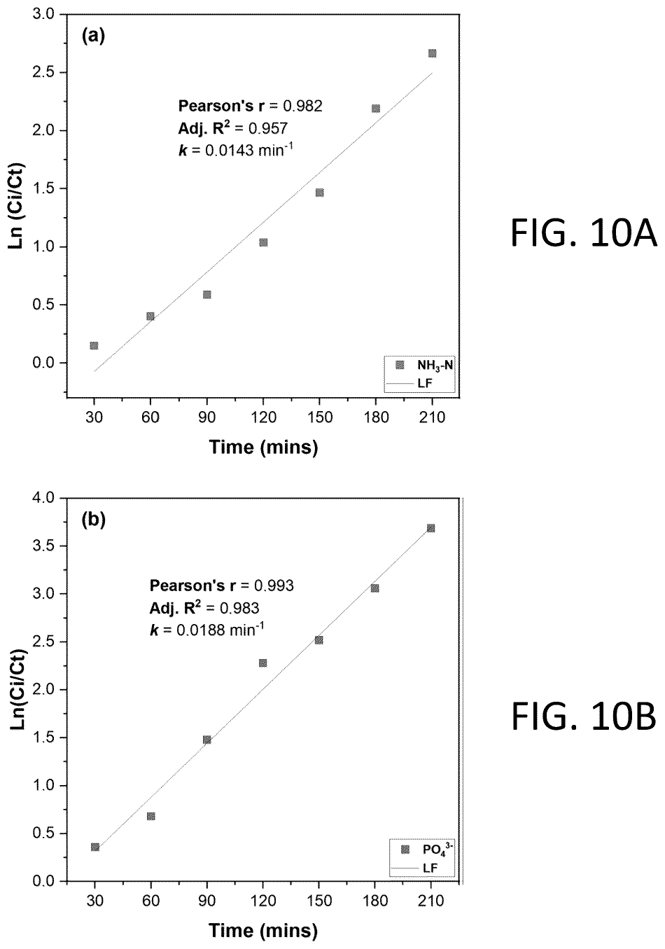

Kinetic modeling was performed for the observed struvite precipitation. The formation of struvite generally proceeds through the following stoichiometric Equation 14:

$${Mg}^{2 +} + {NH}_{{4}^{+}} + {PO}_{4}^{3 -} + 6 {H}_{2} O \to {MgNH}_{4} {PO}_{4} \cdot 6 {H}_{2} O ↓ + {nH}^{+} ↑ \qquad ( Equation 14 )$$

where n=0, 1, or 2, depending on the pH of the solution. It is well-documented that struvite precipitation is a reversible process that follows first-order kinetics. Hence, the kinetic rate constants for PO₄³⁻ and NH₃—N disappearance (−dC/dt) from the solution were determined by fitting integrated forms of the first-order kinetic model to the experimental data. As calculated from the slopes of the logarithmic plots in FIGS. 10A and 10B, the kinetic rate coefficient (k) for PO₄³⁻ and NH₃—N removal were 0.0188 min⁻¹ and 0.0143 min⁻¹, respectively. For both nutrients, the variation of ln (Cᵢ/Cₜ) versus time was almost linear with high correlation coefficient values close to unity; R₂=0.957 for NH₃—N and R₂=0.983 for PO₄³⁻, indicating that the pseudo-first-order kinetic model was appropriate to describe the removal of these nutrients by the ALER system.

It is important to note that since Mg is the limiting reactant, and its dissolution rate depends on the amount of electric current supplied to the system, increasing the current density can speed up the removal of nutrients. The higher the current density, the more Mg²⁺ ions are liberated from the sacrificial anode, increasing the rate of struvite formation with a corresponding decrease in treatment time. However, increasing the current density will also lead to an increase in operating costs.

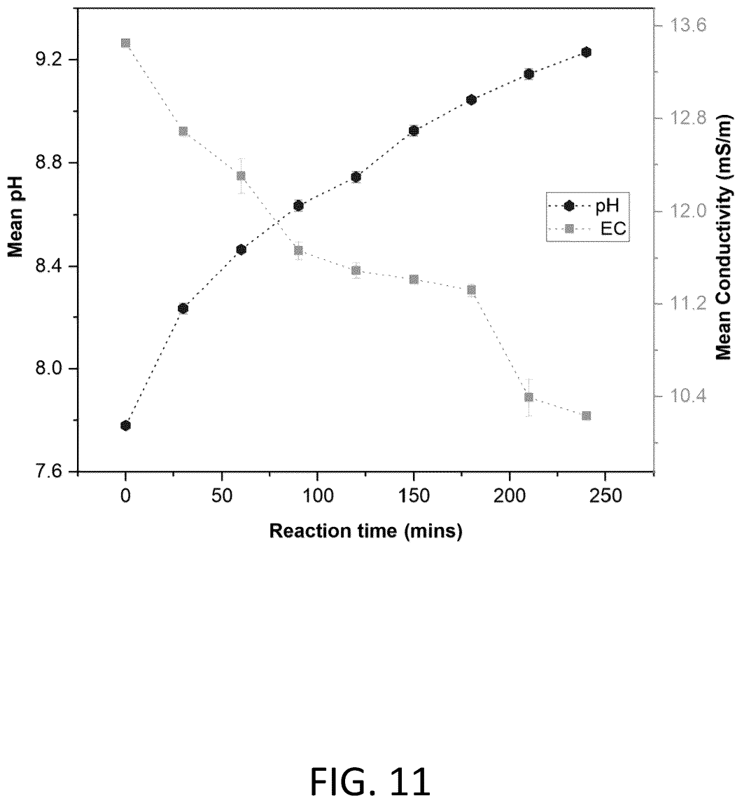

Variation of electrolyte pH and conductivity with treatment time were also observed. The pH value of a wastewater sample has a significant impact on the overall NRE, purity, and growth of struvite crystals inside a reactor since it regulates the solubility and availability or effective concentrations of Mg²⁺, NH₄⁺, and PO₄³⁻, including other constituent ions in the wastewater matrix. FIG. 11 shows the evolution and final pH during a 4 hr batch experiment to recover PO₄³⁻ and NH₃—N from ADPW. The pH of the AD supernatant rose steadily from its initial value of 7.8 to a final value of 9.22 after 4 hrs of treatment. The observed increase in pH can be attributed to the presence of highly reactive hydroxyl radicals (OH⁻) generated at the cathode surface via oxygen reduction or decomposition of water molecules with the evolution of hydrogen gas (H2) according to the following Equations 15 and 16:

$$Cathode [ - ] : {O}_{2} + 2 {H}_{2} O + 4 {e}^{-} \to 4 {OH}^{-} \qquad ( Equation 15 )$$

2H₂O+2e⁻→2H₂↑+2OH⁻ (Equation 16) Many studies have reported that struvite precipitation is possible over a broad range of (4-12). However, a pH range of 8.0-9.5 is recommended to obtain high-purity struvite. In this Example, the initial and final pH values were well within the appropriate pH range to obtain high-purity struvite; hence pH adjustment was unnecessary. This implies that the operating cost can be reduced significantly when the process is scaled-up, considering that calculations from a full-scale pilot plant with a processing capacity of 400 m³/d revealed that the addition of sodium hydroxide for pH regulation was responsible for up to 97% of the overall chemical cost. On the other hand, the electrical conductivity (EC) of the ADPW decreased along with treatment time from its initial value of 13.45 to 10.24 mS/m (23.9%) after 4 hrs. This observation can be attributed to a decrease in total dissolved solids (TDS) and precipitation of minerals as the experiment progressed.

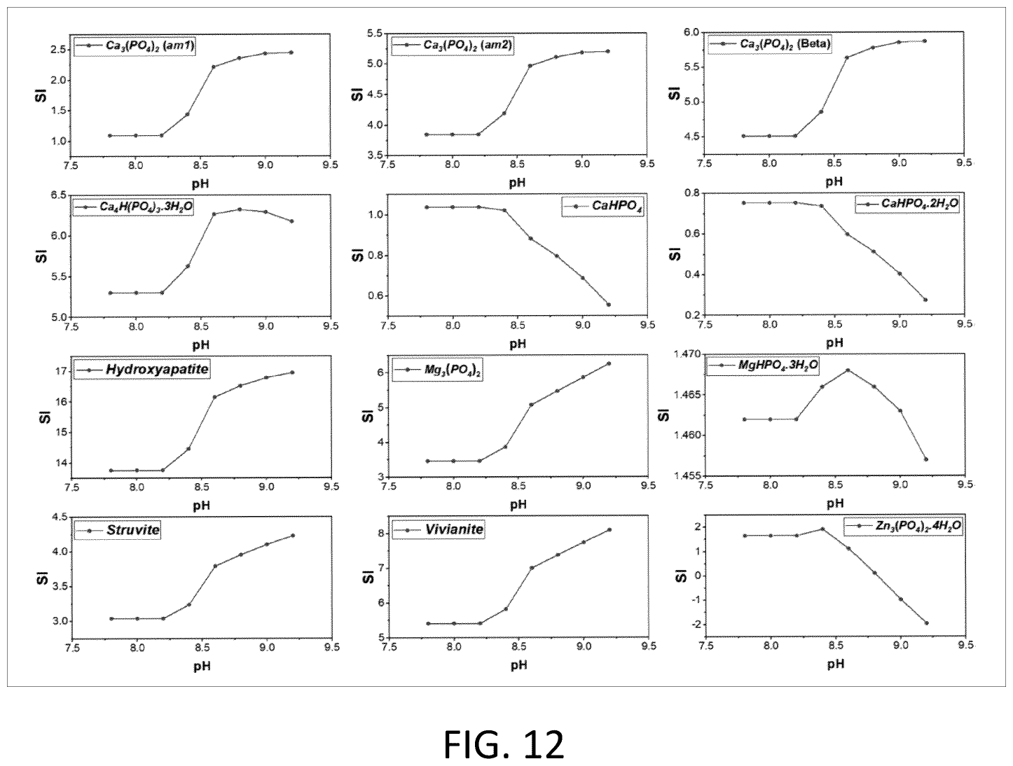

Turning to modeling of thermodynamic chemical equilibrium for the ALER process, for designers and operators, predicting the behavior of nutrient recovery systems or the potential for nutrient recovery from a wastewater sample of known chemical composition under specified operating conditions is essential for anticipating potential mineral formation and unwanted process design failures. The SI is an important parameter that controls crystal nucleation, growth, and aggregation; hence it can be used to assess or predict the likelihood of mineral precipitation and its purity. It is related to the change in the Gibbs free energy (ΔG), which is the thermodynamic driving force for crystal formation and crystal growth, as shown in Equation 17:

$$Δ G = - \frac{2.3 \; 03 RT}{n} \times SI \qquad ( Equation 17 )$$

where T is the temperature, n is the number of ions in the precipitate compound, and R is the universal gas constant. Theoretically, when SI=0, ΔG=0, which denotes equilibrium between solid and liquid phases; while when SI<0, ΔG>0, which suggests unsaturated conditions where no precipitation occurs; and when SI>1, ΔG<0, which indicates supersaturated conditions where spontaneous precipitation occurs. FIG. 12 shows the calculated saturation indices of possible mineral precipitates as a function of pH based on the chemical composition of the ADPW samples used in this Example (see Table 3). A total of 31 different minerals were predicted; however, only 12 were likely to precipitate (SI>1). Based on chemical equilibrium modeling, the 12 possible precipitates were: hydroxyapatite [Ca₅(PO₄)₃OH, HAP], octacalcium phosphate [Ca₄H(PO₄)₃·3H₂O, OCP], magnesium phosphate [Mg₃(PO₄)₂], tricalcium phosphate [am₂-Ca₃(PO₄)₂; am₁-Ca₃(PO₄)₂: β-Ca₃(PO₄)₂], struvite [MgNH₄PO₄·6H₂O], hopeite [Zn₃(PO₄)₂:4H₂O], newberyite [MgHPO₄·3H₂O], vivianite [Fe₃(PO₄)₂·8H₂O], and calcium hydrogen phosphate [CaHPO₄], in the pH range of 7.8-9.2. The data suggest that hydroxyapatite is the least soluble precipitate among all possible precipitates in the system due to its higher SI. The solubility of struvite, HAP, TCP, OCP, Mg₃(PO₄)₂, and vivianite decreased sharply with an increase in pH (over 8.5), while the opposite trend was observed for all other probable precipitates.

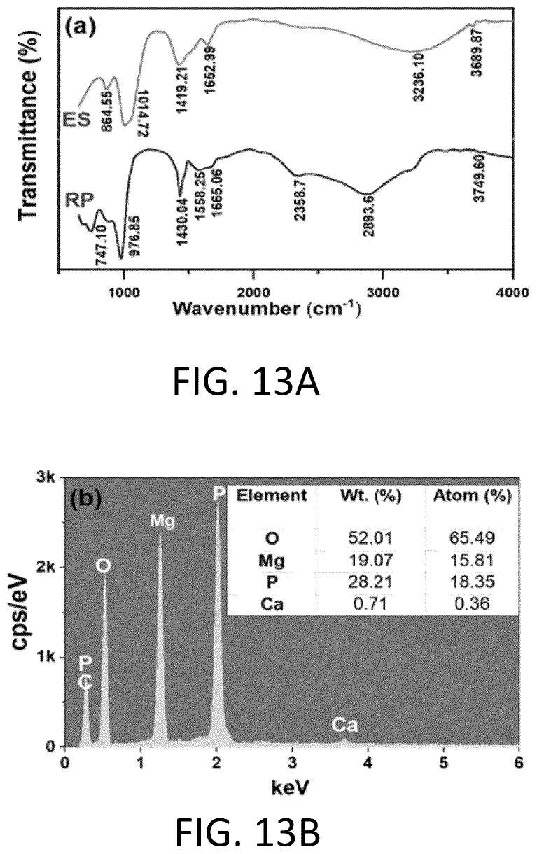

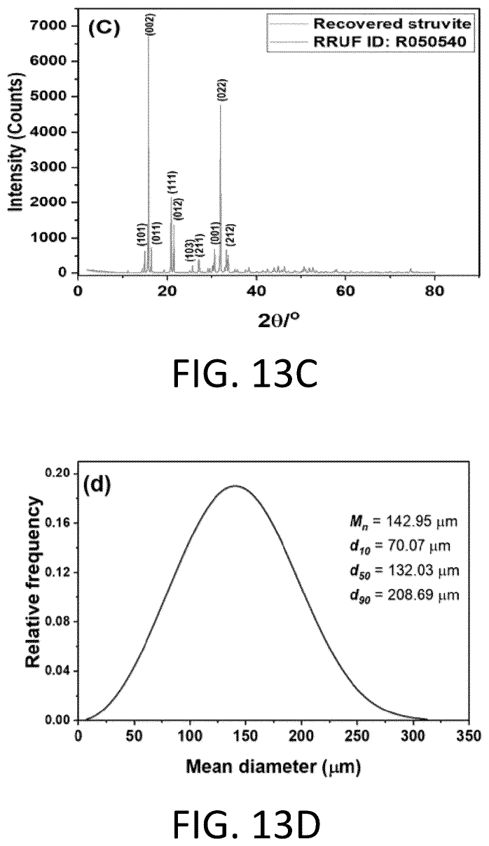

The identification of crystalline phases, elemental composition, and morphology of the precipitates recovered after a 4-hr treatment period were determined using XRD, FTIR, and SEM-EDX, respectively. The XRD spectrum shown in FIG. 13C confirmed the presence of struvite as the main phase formed with characteristic peaks (20) at 15.1°, 16.08°, 21.01°, 21.66°, 25.67°, 27.11°, 30.72°, 31.96° and 33.34° for facets of (101), (002), (011), (111), (012), (103), (211), (001), (022), and (212) respectively, referenced with the JCPDS card (RRUF ID: R050540). No other crystalline phases or minerals were identified by XRD, indicating that the precipitate was high-purity struvite. FT-IR analysis of recovered precipitates showed the infrared spectrum was similar to that of standard struvite pattern, the RP line in FIG. 13A. The band at 1665.06 cm⁻¹ represents the H—O—H deformation of water molecules, while 1430.04 and 1568.25 cm⁻¹ correspond to stretching vibrations of the H—N—H bond in NH₄+. The downward band at 976.85 cm⁻¹ represents the antisymmetric stretching of P—O bonds in PO₄³⁻ while the 2358.7 cm⁻¹ band is associated with water-phosphate hydrogen bonding. The band at 747.10 cm⁻¹ is ascribed to water hydrogen bonding and NH₄—H₂O hydrogen bonding at 864.5 cm⁻¹. The FT-IR and XRD patterns were identical to that of a reference struvite sample (RRUF ID: R050540). FT-IR analysis also revealed that the passivation layer on the anode surface was struvite FIG. 13A (ES). EDS spectrum analysis (FIG. 13B) confirmed the presence of Mg (19.07%), O (52.01%), and P (28.2%) corresponding to struvite composition, together with a small fraction of Ca (0.71%), which indicated that the recovered precipitates were not 100% pure struvite. Ca concentration in the ADPW effluent was 0.2 mg/L; therefore, it is reasonable to assume that there was formation of Ca-containing minerals like HAP or OCP. Ca is an essential element for plant growth, so precipitation of Ca-containing minerals is another phosphorus recovery route. Calcium phosphates were more likely to precipitate than struvite when the Ca/Mg ratio was higher than 1:1. However, in this Example, the concentration of Mg was much higher than that of Ca, which favored more struvite production. The results from this Example also indicated that no heavy metals (Cr, Fe, Cu, Zn, and K) were present in the precipitates recovered after treatment. Coprecipitation of these minerals was not an issue of concern owing to the very low initial concentrations in the liquid phase, which were way lower than the maximum concentrations allowed in organic fertilizers by the U.S. Environmental Protection Agency (USEPA). Acid dissolution of recovered precipitates with subsequent chemical analysis of the NH₃—N content confirmed the presence of struvite as the main phase and revealed that the purity of the crystals was 88.54%.

In nutrient recovery processes, the final PSD is another key indicator of product quality. Since larger crystals are desired (3.5 to 5 mm), it is necessary to have dedicated crystal growth compartments in the crystallizer design. FIG. 13D shows the log-normal (continuous) PSD of precipitates recovered from the ADPW. The data shows a wide distribution of crystals with ferret diameters ranging from 0.1 μm to 258.36 μm with an average size of 142.95 μm and median particle size (D50) of 132.03 μm. The diameters of D10 and D90 were 70.07 and 208.09 μm, respectively. On average, the crystal size of precipitates recovered from the ADPW was smaller than those obtained from simulated poultry wastewater under the same U_{sg} (FIG. 8D). This difference in mean particle size was most likely caused by the difference in wastewater composition. ADPW contains not only large amounts of NH₃—N and PO₄³⁻, including other inorganic ions such as CO₂, Ca²⁺, K⁺ ions, and dissolved solids, but also contains high-strength organics (VFAs, polysaccharides, proteins, humics), and extracellular polymeric substances (EPS) that are either leftover or by-products of COD degradation during the AD process. Several investigations have demonstrated that mere traces of such ionic components in the system can decrease the purity of the final product and also interfere with nucleation and crystal growth kinetics resulting in smaller crystal sizes. As shown in Table 3, the ADPW samples contained 5137 mg/L of COD which could reduce the effective collision of ions and hindered the adsorption of ions on the crystal nucleus. Studies conducted on nutrient recovery through struvite precipitations from anaerobic digestion reactor supernatants have reported varying PSDs. However, because there are very few studies on nutrient recovery from ADPW and because of the high variation of operational parameters, it was challenging to make a direct comparison with PSD from earlier studies. On the positive side, it is possible to obtain larger struvite crystals if the crystals are left in the reactor for much longer. Table 4 shows the performance of similar reactors using a Mg sacrificial anode in terms of NRE, operation conditions (HRT), and the average particle size of recovered solids.

TABLE 4
Comparison of literature on electrochemical nutrient
recovery from different wastewater substrates

| | Type of | | | | | | | |
|---|---|---|---|---|---|---|---|---|
| | substrate | | | | | | | Mean |
| Reactor | treated + | | | | | | | Crystal |
| design and | initial nutrient | Mg | Current | | | | | size |
| volume | concentrations | Anode | density | pH | HRT | NRE | Mixing | (μm) |
| DCE (1 L) | AD Swine | AZ91D | 22.2 | 8.5 | 1 | 93% | 80 | NR |
| with | wastewater | | A/m² | | hr | PO₄₋P | rpm | |
| AEM/CEM | [PO₄³⁻ = 52.8 | | | | | removal | | |
| | mg/L: NH₄⁺ = | | | | | | | |
| | 7.8523 mg/L] | | | | | | | |
| Jar (1.5 L) | Swine | Mg plate | 2 | 7.8 | 45 | 85% | 150 | NR |
| | wastewater | | mA/cm² | | mins | PO₄³⁻ | rpm | |
| | [PO₄³⁻ = 103 | | | | | and | | |
| | mg/L: NH₃₋ | | | | | 90% | | |
| | N = 426 mg | | | | | NH₃₋N | | |
| | L⁻¹] | | | | | removal | | |
| SCR (1 L) | Fermented | AZ91HP | 45 | 7.5-9.3 | 3 | 98% | | NR |
| | waste | | A/m² | | hrs | PO₄⁻P | | |
| | activated | | | | | removal | | |
| | sludge [PO₄⁻P = | | | | | | | |
| | 0.056 g/L: | | | | | | | |
| | NH₃₋N = | | | | | | | |
| | 0.114 g/L] | | | | | | | |
| SCR (0.5 | AD chicken | AZ31B | 20 | 8.6-8.9 | 1.5 | 70.46% | 300 | NR |
| L) | manure slurry | Mg rod | A/cm² | | hrs | PO₄⁻P | rpm | |
| | [PO₄₋P = | | | | | removal | | |
| | 521.50 mg/L: | | | | | | | |
| | NH₃₋N = | | | | | | | |
| | 3994.05 | | | | | | | |
| | mg/L] | | | | | | | |
| CFE (50 L) | Swine | Mg plate | 4 V on | 7.28 | 15 | 99.5% | 20 | 15-20 |
| | wastewater | | 0.04 m² | | mins | PO₄⁻P | rpm | μm |
| | [PO₄₋P | | | | | removal | | |
| | 136.3 mg/L: | | | | | | | |
| | NH₃₋N = 0.41 | | | | | | | |
| | g/L] | | | | | | | |
| Example 2 | AD poultry | AZ91D | 12.4 | 7.8-9.2 | 4 | 99.9% | 9 | 142.95 |
| | [PO₄₋P = | | A/m² | | hrs | PO₄⁻P | L/min | μm |
| | 1397 mg/L: | | | | | and | | |
| | NH₃₋N = | | | | | 97.3% | | |
| | 941 mg/L] | | | | | NH₃₋N | | |
| | | | | | | removal | | |

Operating cost and energy efficiency of the ALER process were evaluated. Large-scale implementation of electrochemical nutrient recovery via struvite precipitation from any type of wastewater depends primarily on its operating cost, including the market price of struvite and other competing fertilizer products. While a complete economic assessment is beyond the scope of this Example, a simplified economic expression can be written if the energy required for mixing is neglected and only the two main components of electrochemical reactor design are considered, as shown in Equation 18:

$$OC ( \$ / {m}^{3} ) = a [ {C}_{energy} ] + b [ {C}_{electrode} ] \qquad ( Equation 18 )$$

where C_{electrode} is the amount of Mg²⁺ consumed for 1 m³ of treated water, C_{energy} is equivalent to the cost of electrical energy consumption per order (EEO) required to treat 1 m³ of wastewater, the coefficient, a, is the cost of electricity ($/kWh) and b is the price of dissolved electrode material ($/kg). The EEO is a powerful scale-up parameter that is of paramount importance in the economic efficiency of electrochemical precipitators employing the use of a Mg sacrificial anode. It is the amount of electricity needed to reduce the concentration of a contaminant by one order of magnitude in a unit volume (1 m³) of wastewater solution. Its value is dependent on both the voltage and amount of the electrical current delivered to the sacrificial electrode according to the following Equation 19:

$${E}_{EO} [ \frac{kWh}{{m}^{3}} ] = \frac{U cell}{V X ln ( Ct / C 0 )} {\int }_{0}^{t} I ( t ) dt \qquad ( Equation 19 )$$

where U_{cell} is the cell voltage (2.12 V), I is the current passed (A); t is the treatment time (4 h), V is the volume of the wastewater treated (0.006 m³), Cₒ and Cₜ are the initial and final concentrations of PO₄³⁻. The EEO of the ALER used in this Example was 0.824 kWh/m³. According to Faraday's law of electrolysis, the quantity of charge supplied to the electrolytic system is directly proportional to the amount of Mg²⁺ ions released from the sacrificial electrode. So, in order to evaluate the consumption of the AZ91 Mg alloy during electrolysis, the amount of Mg²⁺ dissolved (Mₑₓₚ) was measured and compared to the calculated theoretical amount expected to be dissolved (Mₜₕₑₒ) using Faraday's law. At constant current density, assuming that the electrical energy provided was entirely used to oxidize Mg to Mg²⁺, the specific electrical charge (Q) and Mₜₕₑₒ can be obtained using Equations 20 through 22, as follows:

$$Sacrificialanode [ + ] : Mg \to {Mg}^{2 +} + 2 {e}^{-} \qquad ( Equation 20 )$$

$$Q ( \frac{Ah}{{m}^{3}} ) = \frac{I \times t}{3600 \times V} \qquad ( Equation 21 )$$

$${M}_{theo} ( \frac{g}{{m}^{3}} ) = \frac{I \times t \times {M}_{Mg}}{2 \times F \times V} = \frac{3600 \times Q \times {M}_{Mg}}{2 \times F} = 0.4 \; 52 \times Q ( \frac{Ah}{{m}^{3}} ) \qquad ( Equation 22 )$$

where I is the amount of current applied (A); t is the operating time(s); M is the molar mass of magnesium (24.3 g/mol); F is the Faraday constant (96487 C/mol), and V is the volume of solution treated (0.006 m³). Mₑₓₚ was determined by measuring the mass of the AZ91D anode before and after electrolysis. The initial mass of the AZ91D anode was 20.59 g, while the final mass after struvite precipitation was 15.87 g. So, Mₑₓₚ=786.67 g/m³. The faradaic or current efficiency (CE) of the system related to the production of Mg²⁺ ions was calculated using Equation 23:

$$C E ( ∅ ) = \frac{Mexp}{Mtheo} \times 100 \qquad ( Equation 23 )$$

The current efficiency of the ALER was 520.59% which suggested that the actual dissolving rate of the Mg alloy was far greater than its theoretical value. In addition to electrolytic dissolution, chemical dissolution also contributes to the amount of metal ions released from a sacrificial anode. For instance, high PO₄³⁻ concentration can enhance the corrosion rate of Mg electrodes. Therefore, it is plausible that the high PO₄³⁻ concentration in the ADPW caused the AZ91 anode to corrode more quickly, hence the high current efficiency. Considering that only a fraction of the applied current goes towards the desired reaction, the depletion time or lifetime (Dₜ) of the sacrificial AZ91D electrode can be estimated as follows with Equations 24 and 25, assuming that 100% of the electrode is utilized before discarding:

$$ψ C = \frac{n \times F}{{M}_{Mg}} = 7941.15 As / g \qquad ( Equation 24 )$$

$${D}_{t} = \frac{{ψ}_{c} \times {M}_{i}}{I \times ∅} \qquad ( Equation 25 )$$

where Mᵢ is the initial mass of the anode, ψ_{c} is the specific charge capacity of the anode. Under the same experimental conditions, it was estimated that the Mg AZ91 electrode could be used for up to 17.5 hrs. The cost for the amount of Mg consumed was 3.784 $/m³. Therefore, the operating cost for the optimal condition was calculated to be 3.87 $/m³.

### Example 3—Set Up and Results

Another round of anaerobic co-digestion testing was performed using the same set up as Example 2, with a total working volume of 6 L run under constant current density (12 A/m²) and air flow rate (1 L/min). Oxalic acid was used as the extraction acid in place of NaOH to lower the liquid pH to 2.5. The liquid properties before and after treatment are shown in Table 5.

TABLE 5
Acid pre-treated ASBR effluent quality before
and after electrolytic treatment.

| | Before | After | Reduction |
|---|---|---|---|
| Components | (mg/L) | (mg/L) | (%) |
| pH | 7.78 | 8.5 | — |
| Electrical conductivity (mS/cm) | 13.4 | 9.2 | 31.3 |
| Total suspended solids (TSS) | 446.7 | 52.3 | 88.3 |
| Total nitrogen (TN) | 1405.4 | 172 | 87.8 |
| Total phosphorus (TP) | 1397 | Not detected | 100 |
| NH₃—N | 941 | 45 | 95.2 |
| NO₂—N | Not detected | Not detected | — |
| NO₃—N | Not detected | 1.4 | — |
| COD | 5137 | 4539 | 11.6 |
| Mg²⁺ | 195 | 58 | 70.2 |

As indicated in Table 5, for nutrient recovery, TP was completely removed with some NH₃—N and Mg²⁺ remaining. In other words, with the assistance of oxalic acid, the ALER was able to convert all P in the liquid to struvite. It was observed that the solution pH rose quickly to >8 once the electrolytic process started (producing OH—) without needing any chemicals.

In the chemical formula of struvite, MgNH₄PO₄·6H₂O, the molar ratio of the three constituents is 1:1:1. Since nitrogen was not limiting, an appropriate amount of Mg²⁺ equivalent to 0.045 mol of PO₄³⁻ (1397 mg/L of TP=4281 mg/L of PO₄³⁻) was needed. Apparently, the existing amount of Mg²⁺ in the ASBR effluent was not enough (0.195 g/24.3 g/mol=0.008 mol<0.045 mol PO₄³⁻), and the deficiency of Mg²⁺ was then met by the release of Mg²⁺ from the magnesium anode of ALER. In theory, no Mg²⁺ should be left in the effluent, but there was still a small amount of Mg²⁺ (58 mg/L) unused (see Table 5). This observation indicates that the ALER was not optimized, causing over-release of Mg²⁺ from the magnesium anode. Since the amount of Mg²⁺ ions needed in the liquid could be controlled by adjusting the current density applied, to avoid wasting the Mg anode, optimization of the running parameters for the ALER is necessary.

### Example 4—Set Up and Results

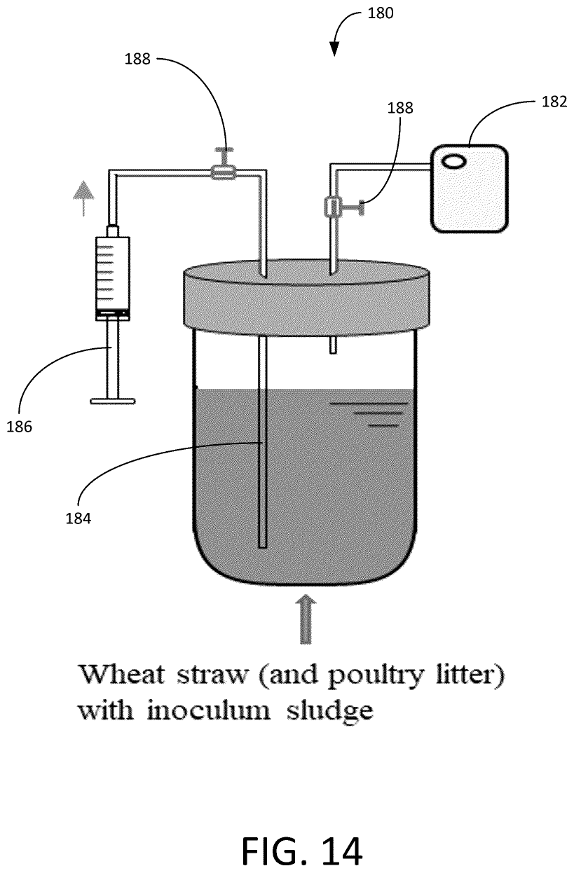

Additional testing was performed to verify the effect of micro-aeration (air injection) on anaerobic co-digestion of poultry litter with wheat straw. Two glass containers 180, each having a total volume of 648 mL (500 mL working volume), were set up for batch operation as shown in FIG. 14. One glass container 180 served as the control, while the other glass container 180 was subjected to micro-aeration treatment. The glass containers 180 were each equipped with a gas outlet for biogas collection in Tedlar® brand gas bags 182 (Tedlar Bag, CEL Scientific Corp., Cerritos, CA, USA). A tubing 184 that reached the sludge at the bottom of the containers 180 was also included for micro-aeration. Mixed substrate of wheat straw and poultry litter, 4.6 g each, was placed in each glass container 180, and each container 180 was then filled with 200 mL inoculum sludge and tap water to make up to the working volume of 500 mL. The glass containers with well-mixed substrate were then sealed and placed in an incubator to maintain temperature at 37° C. throughout the experimental period of 35 days. An air supply rate of 25 mL air/Lr/day (where Lr refers to reactor working volume) was used for the glass container 180 subjected to micro-aeration treatment. The air supply rate was controlled using the combination of a syringe 186 and a gas valve 188 connected to the tubing 184. At the end of test, cumulative methane volumes of 225.44 mL and 181.44 mL were obtained from the air treated and control containers 180, respectively, showing that the former enhanced the total volume of CH₄ production by 24.25% over the latter. These results strongly evidence that micro-aeration improves the performance of the anaerobic co-digestion process in terms of methane production.

For purposes of the instant disclosure, the term “at least” followed by a number is used herein to denote the start of a range beginning with that number (which may be a range having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted as a range whose lower limit is 25 and whose upper limit is 100. Additionally, it should be noted that where a range is given, every possible subrange or interval within that range is also specifically intended unless the context indicates the contrary. For example, if the specification indicates a range of 25 to 100, such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only, and decimal and fractional values (e.g., 46.7-91.3) should also be understood to be intended as possible subrange endpoints unless specifically excluded.

It should be understood that the exemplary embodiments described above should be considered in a descriptive sense only and not for purposes of limitation. Descriptions of features or aspects within these embodiments should typically be considered as available for other similar features or aspects in other embodiments.

While one or more embodiments have been described with reference to the figures, it will be understood by those of ordinary skill in the art that various changes in form and details may be made therein without departing from the scope of the inventive concept as defined by the following claims.

What is claimed is:

1. A poultry litter digester comprising:
an anaerobic digestion reactor configured to digest poultry litter to produce an effluent and a biogas; and
an electrolytic reactor comprising at least one magnesium anode, wherein the electrolytic reactor is configured to precipitate struvite from the effluent.

2. The poultry litter digester of claim 1, wherein the effluent comprises Mg²⁺, NH₄⁺, and PO₄³.

3. The poultry litter digester of claim 1, wherein the biogas comprises methane.

4. The poultry litter digester of claim 1, wherein the anaerobic digestion reactor is an anaerobic sequencing batch reactor, a plug flow reactor, an induced blanket reactor, an up flow anaerobic sludge blanket, a continuous stirred tank reactor, or an anaerobic filter.

5. The poultry litter digester of claim 4, wherein the anaerobic digestion reactor is an anaerobic sequencing batch reactor.

6. The poultry litter digester of claim 5, wherein the anaerobic sequencing batch reactor is a liquid anaerobic sequencing batch reactor that is configured to perform micro-aeration.

7. The poultry litter digester of claim 1, wherein the electrolytic reactor is a column air-lift electrolytic reactor or a dual-chamber reactor.

8. The poultry litter digester of claim 7, wherein the electrolytic reactor is a column air-lift electrolytic reactor.

9. The poultry litter digester of claim 1, wherein the electrolytic reactor is further configured to produce a rejected water as the struvite is precipitated.

10. The poultry litter digester of claim 9, further comprising:
a water reclamation system configured to convert the rejected water from the electrolytic reactor into reclaimed water; and
at least one pump configured to transfer the reclaimed water to the anaerobic digestion reactor.

11. The poultry litter digester of claim 10, wherein the water reclamation system is a forward osmosis reactor.

12. A method for digesting poultry litter comprising the steps of:
preparing a substrate solution from poultry litter and a carbon-rich source;
introducing the substrate solution into an anaerobic digestion reactor;
performing anaerobic co-digestion on the substrate solution within the anaerobic digestion reactor to recover an effluent and a biogas; and
routing the effluent to an electrolytic reactor, wherein the electrolytic reactor is configured to precipitate struvite from the effluent.

13. The method of claim 12, wherein the step of performing anaerobic co-digestion on the substrate solution further comprises a step of introducing air or oxygen to the anaerobic digestion reactor.

14. The method of claim 13, wherein the air or oxygen is introduced to the anaerobic digestion reactor at an air supply rate of 25 mL per L of reactor working volume per day.

15. The method of claim 12, wherein the electrolytic reactor is further configured to produce a rejected water as the struvite is precipitated.

16. The method of claim 15, further comprising the steps of:
recycling the rejected water into a reclaimed water; and
pumping the reclaimed water to the anaerobic digestion reactor.

17. The method of claim 12, further comprising the step of drying the struvite.

18. A poultry litter digester comprising:
an anaerobic digestion reactor configured to digest poultry litter to produce an effluent and a biogas;
an electrolytic reactor comprising at least one magnesium anode, wherein the electrolytic reactor is configured to precipitate struvite from the effluent while producing a rejected water; and
a water reclamation system configured to convert the rejected water from the electrolytic reactor into reclaimed water.

19. The poultry litter digester of claim 18, wherein the anaerobic digestion reactor is configured to perform micro-aeration.

20. The poultry litter digester of claim 18, wherein the anaerobic digestion reactor is an anaerobic sequencing batch reactor, and the electrolytic reactor is a column air-lift electrolytic reactor.
